(19) United States
(12) Patent Application Publication
(10) Pub. No.: US 2025/0362278 A1
(43) Pub. Date: Nov. 27, 2025

(54) MASS SPECTROMETRY OF MRNA

(71) Applicant: Moderna TX, Inc., Cambridge, MA (US)

(72) Inventors: Eva-Maria Schneeberger, Cambridge, MA (US); Tao Jiang, Quincy, MA (US)

(73) Assignee: ModernaTX, Inc., Cambridge, MA (US)

(21) Appl. No.: 18/706,043

(22) PCT Filed: Oct. 31, 2022
(86) PCT No.: PCT/US2022/048385

§ 371 Date: Apr. 30, 2024

Related U.S. Application Data
(60) Provisional application No. 63/274,155, filed on Nov. 1, 2021.

(51) Int. Cl. G01N 30/72; G01N 30/02; G01N 30/30; G01N 30/88

(52) U.S. Cl. CPC G01N 30/724; G01N 2030/027; G01N 2030/3007; G01N 2030/8827

(57) ABSTRACT

Provided herein are methods of determining the size and purify of nucleic acids (e.g., mRNAs) by using hydrophilic interaction chromatography (HILIC)-based methods to separate the nucleic acids from a mixture, followed by mass spectrometry to determine the size of the nucleic acids.

### RELATED APPLICATION

This application claims the benefit under 35 U.S.C. § 119(e) of the earlier filing dates of U.S. Provisional Application No. 63/274,155, filed Nov. 1, 2021, the contents of which are incorporated by reference herein in their entirety.

### BACKGROUND

Recently, messenger ribonucleic acid (mRNA)-based therapeutics have shown promise as vaccines for infectious diseases, such as SARS-CoV-2, with the added ability to quickly adapt to viral mutations. Critical to the development of mRNA drugs is a thorough understanding of their critical quality attributes, such as capping efficiency, tail integrity, sequence identity, and integrity. Several methods of analyzing the purity of polynucleotides are known, including capillary electrophoresis, gel electrophoresis, and mass spectrometry. However, such methods are generally limited to analysis of small RNAs (<200 nt in length), as resolution is poor when analyzing longer RNAs.

### SUMMARY

Provided herein are methods of analyzing compositions containing nucleic acids (e.g., mRNAs) by using hydrophilic interaction chromatography (HILIC)-based methods to purify one or more nucleic acids from a composition, then using mass spectrometry to determine the mass of the purified nucleic acid. Longer nucleic acids contain more sites that can be deprotonated, and more deprotonated sites impart a higher charge state to the nucleic acid. These higher charge states cause merging of individual mass-to-charge peaks in spectra generated by mass spectrometry, which reduces the ability of mass spectrometry to resolve the mass of larger nucleic acids. Surprisingly, purifying nucleic acids by hydrophilic interaction chromatography reduced the average charge state of the purified nucleic acids, which improved the resolution of mass spectra. Mass spectrometry was able to accurately determine the mass of nucleic acids longer than 2,000 nucleotides, a substantial improvement over previous methods, which were typically limited to analysis of nucleic acids shorter than 300 nucleotides.

Accordingly, the present disclosure provides, in some aspects, a method of identifying a target mRNA in a mixture, the method comprising:
(i) contacting a stationary phase of a hydrophilic interaction chromatography (HILIC) column with one or more mRNAs;
(ii) detecting a signal corresponding to the retention time of the target mRNA;
(iii) eluting the target mRNA from the HILIC column; and
(iv) determining the mass of the eluted mRNA of (iii) using mass spectrometry.

In some embodiments, the method further comprises contacting the column with a mobile phase comprising a first solvent solution and a second solvent solution each comprising at least one ion pairing agent, and wherein the first solvent solution further comprises at least about 50% v/v of an organic solvent, such that the target mRNA traverses the column with a retention time that is characteristic of the target mRNA.

In some embodiments, the first solvent solution and second solvent solution each comprise at least two ion pairing agents in a molar ratio of between about 1:10 to about 10:1. In some embodiments, the first and/or second solvent solution are in a molar ratio between about 1:4 to about 4:1, about 1:5 to about 5:1, about 1:5 to about 5:1, about 1:3 to about 3:1, about 1:2 to about 2:1, or about 1:1.5 to about 1.5:1. In some embodiments, the at least two ion pairing agents in the first and/or second solvent solution are in a 1:1 molar ratio.

In some embodiments, the at least one ion pairing agent in the first and/or second solvent solution is selected from the group consisting of a trietheylammonium salt, tributylammonium salt, hexylammonium salt, dibutylammonium salt, tetrapropylammonium salt, dodecyltrimethylammonium salt, tetra(decyl)ammonium salt, dihexylammonium salt, dipropylammonium salt, myristyltrimethylammonium salt, tetraethylammonium salt, tetraheptylammonium salt, tetrahexylammonium salt, tetrakis(decyl)ammonium salt, tetramethylammonium salt, tetraoctylammonium salt, and tetrapentylammonium salt, optionally wherein the triethylammonium salt is triethylammonium acetate, the tributylammonium salt is tetrabutylammonium phosphate or tetrabutylammonium chloride, the hexylammonium salt is hexylammonium acetate, the dibutylammonium salt is dibutylammonium acetate, the tetrapropylammonium salt is dodecyltrimethylammonium chloride, the tetra(decyl)ammonium salt is tetra(decyl)ammonium bromide, the dihexylammonium salt is dihexylammonium acetate, the dipropylammonium salt is dipropylammonium acetate, the myristyltrimethylammonium salt is myristyltrimethylammonium bromide, the tetraethylammonium salt is tetraethylammonium bromide, the etraheptylammonium salt is tetraheptylammonium bromide, the tetrahexylammonium salt is tetrahexylammonium bromide, the tetrakis(decyl)ammonium salt is tetrakis(decyl)ammonium bromide, the tetramethylammonium salt is tetramethylammonium bromide, the tetraoctylammonium salt is tetraoctylammonium bromide, and/or the tetrapentylammonium salt is tetrapentylammonium bromide.

In some embodiments, the first solvent solution and the second solvent solution each comprise at least two ion pairing agents, wherein the at least two ion pairing agents are (i) octylamine and nonafluoro-tert-butyl alcohol; (ii) octylamine and diethylammonium acetate; (iii) octylamine and dibutylammonium acetate; or (iv) diethylammonium acetate and imidazole.

In some embodiments, the concentration of each of the at least one ion pairing agents in the first solvent solution and/or the second solvent solution ranges from about 10 mM-20 M, 20 mM-15 M, 30 mM-12 M, 40 mM-10 M, 50 mM-8 M, 75 mM-5 M, 100 mM-2.5 M, 125 mM-2 M, 150 mM-1.5 M, 175 mM-1 M, or 200 mM-500 mM,

optionally wherein the concentration of each of the at least one ion pairing agents in the first solvent solution and/or the second solvent solution ranges from about 10 mM-1M, 40 mM-300 mM, 50 mM-500 mM, 75 mM-400 mM, 100 mM-300 mM, 200-300 mM, 200-250 mM, or 250-300 mM.

In some embodiments, the first solvent solution comprises about 50% to about 95%, about 55% to about 90%, about 60% to about 85%, about 65% to about 80%, or about 70% v/v to about 75% v/v of the organic solvent,

optionally wherein the first solvent solution comprises about 50%, about 60%, about 70%, about 80%, or about 90% v/v of the organic solvent.

In some embodiments, the organic solvent in the first solvent solution is selected from the group consisting of polar aprotic solvents, C₁₋₄ alkanols, C₁₋₆ alkanediols, and C₂₋₄ alkanoic acids.

In some embodiments, the organic solvent in the first solvent solution is selected from the group consisting of acetonitrile, methanol, ethanol, isopropanol, acetone, propanol, tetrahydrofuran, dimethyl sulfoxide, dimethylformamide, and hexylene glycol.

In some embodiments, the pH of the first solvent solution and/or the second solvent solution is between about pH 6.5 and pH 9.0.

In some embodiments, the volume percentage of the first solvent solution and volume percentage of the second solvent solution in the mobile phase are each varied between 0% and 100%.

In some embodiments, the ratio of the first solvent solution to the second solvent solution is held constant during elution of the mRNA.

In some embodiments, the ratio of the first solvent solution to the second solvent solution is increased or decreased during elution of the mRNA.

In some embodiments, the concentration of each ion pairing agent in the mobile phase is held constant during elution of the mRNA.

In some embodiments, the concentration of one or more ion pairing agents in the mobile phase is increased or decreased during elution of the mRNA.

In some embodiments, the eluting is gradient or isocratic with respect to the concentration of the organic solvent.

In some embodiments, each of the first and second solvent solutions comprises one or more volatile salts.

In some embodiments, the at least one volatile salt in the first and/or second solvent solution is selected from the group consisting of formic acid, acetic acid, trifluoroacetic acid, ammonium formate, ammonium acetate, ammonium hydroxide, triethylamine acetate, triethylamine formate, diethylamine acetate, diethylamine formate, piperidine acetate, piperidine formate, ammonium bicarbonate, borate, hydride, 4-methylmorpholine, 1-methylpiperidine, pyrrolidine acetate, and pyrrolidine formate.

In some embodiments, the concentration of each of the at least one volatile salts in the first solvent solution and/or the second solvent solution ranges from about 10 mM-20 M, 20 mM-15 M, 30 mM-12 M, 40 mM-10 M, 50 mM-8 M, 75 mM-5 M, 100 mM-2.5 M, 125 mM-2 M, 150 mM-1.5 M, 175 mM-1 M, or 200 mM-500 mM,

optionally wherein the concentration of each of the at least one volatile salts in the first solvent solution and/or the second solvent solution ranges from about 10 mM-1M, 40 mM-300 mM, 50 mM-500 mM, 75 mM-400 mM, 100 mM-300 mM, 200-300 mM, 200-250 mM, or 250-300 mM.

In some embodiments, the column is an analytical column, or a preparative column.

In some embodiments, the stationary phase comprises particles.

In some embodiments, the particles have a diameter of about 2 μm-about 10 μm, about 2 μm-about 6 μm, or about 4 μm

In some embodiments, the particles are porous resin particles, optionally wherein the particles comprise pores having a diameter of about 500 Å to about 5000 Å, about 800 Å to about 3000 Å, or about 1000 Å to about 2000 Å.

In some embodiments, the stationary phase is hydrophilic or comprises hydrophilic functional groups.

In some embodiments, the column has a temperature from about 20° C. to about 60° C.

In some embodiments, the method has a run time of between about 10 minutes and about 30 minutes.

In some embodiments, the target mRNA is present in a composition added to the column in an amount ranging from about 0.05 mg/mL to about 1 mg/mL, optionally wherein the amount is 0.1 mg/mL.

In some embodiments, determining the mass of the eluted mRNA using mass spectrometry comprises using MALDI and/or ESI to ionize the mRNA, followed by TOF mass spectrometry to analyze the ionized mRNA.

In some embodiments, the target mRNA is single-stranded.

In some embodiments, the target mRNA comprises:
(i) 5′ and 3′ untranslated regions (UTRs);
(ii) a 5′ cap, optionally wherein the 5′ cap is a 7-methylguanosine cap or a 7-methylguanosine group analog; and
(iii) a 3′ polyadenosine (polyA) tail.

In some embodiments, the target mRNA is a linear mRNA.

In some embodiments, the target mRNA is a circular mRNA.

In some embodiments, the circular mRNA comprises an internal ribosome entry site (IRES).

In some embodiments, the circular mRNA comprises in 5′ to 3′ order, a 5′ untranslated region (UTR), an IRES, an open reading frame encoding a protein, and a 3′ untranslated region.

In some embodiments, the circular RNA further comprises a poly(A) region.

In some embodiments, the poly(A) region is between the 5′ UTR and the IRES.

In some embodiments, the poly(A) region is between the open reading frame and the 3′ UTR.

In some embodiments, the mRNA is an in vitro transcribed (IVT) mRNA.

In some embodiments, the mRNA encodes a vaccine antigen or therapeutic polypeptide.

In some embodiments, the target mRNA has a length between 300-500 nucleotides, 500-1000 nucleotides, 1000-1500 nucleotides, 1500-2000 nucleotides, 2000-2500 nucleotides, 2500-3000 nucleotides, 3000-3500 nucleotides, 3500-4000 nucleotides, 4000-4500 nucleotides, or 4500-5000 nucleotides.

### BRIEF DESCRIPTION OF THE DRAWINGS

FIG. 1 shows the effects of nucleic acid charge state distributions on mass-to-charge spectra obtained by mass spectrometry.

FIGS. 2A-2B show the effects of volatile salts on mass spectrometry analysis of mRNAs purified by HILIC-based methods. FIG. 2A shows UV chromatograms indicating the retention time of an mRNA in a HILIC column when mobile phases contained volatile salts of bicarbonate, acetate, or formate. FIG. 2B shows mass-to-charge spectra when mRNAs obtained by HILIC-based purification using mobile phases that contained bicarbonate, acetate, or formate.

FIGS. 3A-3B show the effects of organic solvents on mass spectrometry analysis of mRNAs purified by HILIC-based methods. FIG. 3A shows UV chromatograms indicating the retention time of an mRNA in a HILIC column when mobile phases contained organic solvents of methanol, acetonitrile, or isopropanol. FIG. 3B shows mass-to-charge spectra when mRNAs obtained by HILIC-based purification using mobile phases that contained methanol, acetonitrile, or isopropanol.

FIG. 4 shows the effects of ion pairing agents on mass spectrometry analysis of mRNAs purified by HILIC-based methods. Mass-to-charge spectra are shown for mRNAs obtained by HILIC-based purification using mobile phases that contained ion pairing agents of (i) octylamine and nonafluoro-tert-butyl alcohol; (ii) diethylammonium acetate; or (iii) dibutylammonium acetate.

FIGS. 5A-5C show analysis of poly-A tail heterogeneity by LC/MS-based methods. FIG. 5A shows a mass-to-charge spectrum of a tailless mRNA, produced by RNase H cleavage of the poly-A tail from the mRNA analyzed in FIGS. 2A-4. FIG. 5B shows a zoomed-in spectra from FIG. 5A, indicating distinct, resolvable peaks corresponding to distinct ion species. FIG. 5C shows a deconvoluted mass spectrum with distinct peaks separated by approximately 329 Da, the mass of an adenosine residue, corresponding to mRNAs with different poly-A tail lengths.

FIG. 5D shows a series of secondary peaks, each 135 Da less than a corresponding main peak, consistent with loss of the adenine base from a nucleotide of the poly-A tail.

FIGS. 6A-6D show mass-to-charge spectra of four tailless mRNA species having lengths of 751 nt (FIG. 6A), 1822 nt (FIG. 6B), 1894 nt (FIG. 6C), and 2228 nt (FIG. 6D). Insets show deconvoluted mass spectra, with estimated mRNA mass and estimation error.

FIGS. 7A-7E show analysis of circular mRNA by LC/MS-based methods. FIG. 7A shows a mass-to-charge spectrum of a first circular mRNA. FIG. 7B shows a deconvoluted mass spectrum with a main peak corresponding to the theoretical mass of the first circular mRNA. FIG. 7C shows an enlarged portion of the mass spectrum of FIG. 7B, with distinct peaks separated by approximately 329 Da, the mass of an adenosine residue, corresponding to mRNAs with poly-A regions of different lengths. FIGS. 7D and 7E show similar analyses as FIGS. 7B-7C, for a second circular mRNA encoding a different protein and having a different mass.

### DETAILED DESCRIPTION

Provided herein are methods of analyzing compositions containing nucleic acids (e.g., mRNAs) by using hydrophilic interaction chromatography (HILIC)-based methods to purify one or more nucleic acids from a composition, then using mass spectrometry to determine the mass of the purified nucleic acid. Longer nucleic acids contain more sites that can be deprotonated, and more deprotonated sites impart a higher charge state to the nucleic acid. These higher charge states cause merging of individual mass-to-charge peaks in spectra generated by mass spectrometry, which reduces the ability of mass spectrometry to resolve the mass of larger nucleic acids. Surprisingly, purifying nucleic acids by hydrophilic interaction chromatography reduced the average charge state of the purified nucleic acids, which improved the resolution of mass spectra. Mass spectrometry was able to accurately determine the mass of nucleic acids longer than 2,000 nucleotides, a substantial improvement over previous methods, which were typically limited to analysis of nucleic acids shorter than 300 nucleotides.

### Hydrophilic Interaction Chromatography (HILIC) Methods

Aspects of the present disclosure relate to methods of separating one or more nucleic acids (e.g., mRNAs) from a mixture using hydrophilic interaction chromatography (HILIC). Generally, a HILIC column comprises a polar stationary phase with an affinity for polar analytes. A mixture comprising one or more polar analytes to be separated from the mixture is added to the polar stationary phase of the column, and a mobile phase comprising an alcohol and/or aprotic solvent is also added to the column to promote passage of the analytes through the stationary phase. Without wishing to be bound by theory, it is believed that any water present in the mobile phase associates with the polar stationary phase, increasing the affinity of polar analytes such as nucleic acids for the stationary phase. More polar analytes, such as longer nucleic acids, have stronger affinities for the stationary phase, and are thus retained in the column for longer, thereby allowing HILIC methods to separate nucleic acids by length. HILIC methods using a mobile phase that is compatible with downstream applications such as mass spectrometry allow the mass of the purified nucleic acid(s) to be analyzed by mass spectrometry.

In some instances, the methods of the disclosure are used to determine the identity, stability or integrity of a nucleic acid, such as a target nucleic acid, in a composition. In some instances, the methods of the disclosure are used to determine the purity of a nucleic acid, such as a target nucleic acid, in a composition. As used herein, the term “target nucleic acid” refers to a nucleic acid of interest, the presence, abundance, and/or purity of which may be measured by any of the methods provided herein. In some embodiments, the term “target mRNA” refers to a target nucleic acid that is an mRNA. In some embodiments, the methods provided herein specifically quantify the presence, abundance, and/or purity of one or more target nucleic acids (e.g., target mRNAs) in a composition. Specifically quantifying a characteristic of a target nucleic acid refers to measuring the characteristic with respect to that nucleic acid species (e.g., mRNA containing a particular open reading frame sequence), rather than the measuring the characteristic with respect to all nucleic acids in the composition. For example, specifically quantifying the abundance of a target nucleic acid refers to quantifying the amount of the target nucleic acid in the composition, irrespective of the total abundance of all nucleic acids in the composition. As used herein, the term “pure” refers to material that has only the target nucleic acid active agents such that the presence of unrelated nucleic acids is reduced or eliminated, e.g., impurities or contaminants, including nucleic acid fragments. For example, a purified RNA sample includes one or more target or test nucleic acids but is preferably substantially free of other nucleic acids. As used herein, the term “substantially free” is used operationally, in the context of analytical testing of the material. Preferably, purified material substantially free of impurities or contaminants is at least 95% pure; more preferably, at least 98% pure, and more preferably still at least 99% pure. In some embodiments a pure nucleic acid sample is comprised of 100% of the target or test nucleic acids and includes no other nucleic acids. In some embodiments, it only includes a single type of target or test nucleic acid. In some embodiments a pure RNA sample is comprised of 100% of the target or test RNAs and includes no other RNA. In some embodiments it only includes a single type of target or test RNA.

A “reference nucleic acid” as used herein refers to a control nucleic acid (e.g., a non-target nucleic acid) or chromatogram generated from a control nucleic acid that uniquely identifies the nucleic acid separated from the mixture. The reference nucleic acid may be generated based on digestion of a pure sample and compared to data generated by HPLC of a composition comprising the nucleic acid of interest (e.g., a target nucleic acid, such as a target mRNA). Alternatively, it may be a known chromatogram, stored in an electronic or non-electronic data medium. For example, a control chromatogram may be a chromatogram based on predicted HPLC retention times of a particular RNA (e.g., a test mRNA). In some embodiments, quality control methods described by the disclosure further comprise the step of comparing the nucleic acid separated from the mixture to the reference nucleic acid using an orthogonal analytical technique, for example polymerase chain reaction (e.g., RT-qPCR), nucleic acid sequencing, gel electrophoresis, and/or mass spectrometry.

Accordingly, in some aspects, the present disclosure provides methods of identifying a target mRNA in a mixture, the method comprising:
(i) contacting a stationary phase of a hydrophilic interaction chromatography (HILIC) column with one or more mRNAs;
(ii) detecting a signal corresponding to the retention time of the target mRNA;
(iii) eluting the target mRNA from the HILIC column; and
(iv) determining the mass of the eluted mRNA of (iii) using a mass spectrometer.

In some embodiments, the method further comprises contacting the HILIC column with a mobile phase comprising a first solvent solution and a second solvent solution each comprising at least one ion pairing agent, such that the target mRNA traverses the column with a retention time that is characteristic of the target mRNA.

In some embodiments, one or more solvent solutions (e.g., 1, 2, 3, 4, 5, or more) of the mobile phase comprise a combination of at least two ion pairing agents (e.g., 2, 3, 4, 5, or more). As used herein, an “ion pairing agent” or an “ion pair” refers to an agent (e.g., a small molecule) that functions as a counter ion to a charged (e.g., ionized or ionizable) functional group on an analyte (e.g., a nucleic acid) and thereby changes the retention time of the analyte as it moves through the stationary phase of a column. Generally, ion paring agents are classified as cationic ion pairing agents (which interact with negatively charged functional groups) or anionic ion pairing agents (which interact with positively charged functional groups). The terms “ion pairing agent” and “ion pair” further encompass an associated counter-ion (e.g., acetate, phosphate, bicarbonate, bromide, chloride, citrate, nitrate, nitrite, oxide, sulfate and the like, for cationic ion pairing agents, and sodium, calcium, and the like, for anionic ion pairing agents). In some embodiments, one or more ion pairing agents utilized in the methods described by the disclosure is a cationic ion pairing agent. Examples of cationic ion pairing agents include but are not limited to certain protonated or quaternary amines (including e.g., primary, secondary and tertiary amines) and salts thereof, such as a trietheylammonium salt (e.g., triethylammonium acetate (TEAA)), a tributylammonium salt (e.g., tetrabutylammonium phosphate (TBAP) or tetrabutylammonium chloride (TBAC)), a hexylammonium salt (e.g., hexylammonium acetate (HAA)), a dibutylammonium salt (e.g., dibutylammonium acetate (DBAA)), a tetrapropylammonium salt (e.g., tetrapropylammonium bromide (TPAB)), a dodecyltrimethylammonium salt (e.g., dodecyltrimethylammonium chloride (DTMAC)), or a tetra(decyl)ammonium salt (e.g., tetra(decyl)ammonium bromide (TDAB)), a dihexylammonium salt (e.g., dihexylammonium acetate (DHAA)), a dipropylammonium salt (e.g., dipropylammonium acetate (DPAA)), a myristyltrimethylammonium salt (e.g., myristyltrimethylammonium bromide (MTEAB)), a tetraethylammonium salt (e.g., tetraethylammonium bromide (TEAB)), a tetraheptylammonium salt (e.g., tetraheptylammonium bromide (THepAB)), a tetrahexylammonium salt (e.g., tetrahexylammonium bromide (THexAB)), a tetrakis(decyl)ammonium salt (e.g., tetrakis(decyl)ammonium bromide (TrDAB)), a tetramethylammonium salt (e.g., tetramethylammonium bromide (TMAB)), a tetraoctylammonium salt (e.g., tetraoctylammonium bromide (TOAB)), or a tetrapentylammonium salt (e.g., tetrapentylammonium bromide (TPeAB)). Other examples of ion pairing agents include imidazoles, imidazolium salts (e.g., imidazolium acetate), propylamine, butylamine, pentylamine, hexylamine, hepyltamine, and octylamine, fluoroalcohols (e.g., trifluoroethanol, hexafluoroisopropanol, perfluoro-tert-butanol, and nonafluoro-tert-butyl alcohol). In some embodiments, one or more solvent solutions of the mobile phase comprise a combination of two or more ion pairing agents selected from the group consisting of an imidazole, an imidazolium salt, propylamine, butylamine, pentylamine, hexylamine, hepyltamine, octylamine, trifluoroethanol, hexafluoroisopropanol, perfluoro-tert-butanol, nonafluoro-tert-butyl alcohol, a trietheylammonium salt, tributylammonium salt, hexylammonium salt, dibutylammonium salt, tetrapropylammonium salt, dodecyltrimethylammonium salt, tetra(decyl)ammonium salt, dihexylammonium salt, dipropylammonium salt, myristyltrimethylammonium salt, tetraethylammonium salt, tetraheptylammonium salt, tetrahexylammonium salt, tetrakis(decyl)ammonium salt, tetramethylammonium salt, tetraoctylammonium salt, and tetrapentylammonium salt. In some embodiments, one or more solvent solutions of the mobile phase comprise a combination of two or more ion pairing agents selected from the group consisting of an imidazole, an imidazolium salt, propylamine, butylamine, pentylamine, hexylamine, hepyltamine, octylamine, trifluoroethanol, hexafluoroisopropanol, perfluoro-tert-butanol, nonafluoro-tert-butyl alcohol, HAA, TBAP, TPAB, TBAC, DBAA, TEAA, DTMAC, TDAB, DHAA, DPAA MTEAB, TEAB, THepAB, THexAB, TrDAB, TMAB, TOAB, and TPeAB. In some embodiments, one or more solvent solutions of the mobile phase comprise a combination of (i) octylamine and nonafluoro-tert-butyl alcohol; (ii) octylamine and diethylammonium acetate; (iii) octylamine and dibutylammonium acetate; or (iv) diethylammonium acetate and imidazole. In some embodiments, one or more solvent solutions of the mobile phase comprise a combination of octylamine and nonafluoro-tert-butyl alcohol. In some embodiments, one or more solvent solutions of the mobile phase comprise a combination of octylamine and diethylammonium acetate. In some embodiments, one or more solvent solutions of the mobile phase comprise a combination of octylamine and dibutylammonium acetate. In some embodiments, one or more solvent solutions of the mobile phase comprise a combination of diethylammonium acetate and imidazole.

In some embodiments, one or more solvent solutions (e.g., 1, 2, 3, 4, 5, or more) of the mobile phase comprise a single ion pairing agent. In some embodiments, one or more ion pairing agents utilized in the methods described by the disclosure is a cationic ion pairing agent. In some embodiments, the ion pairing agent is a cationic ion pairing agent. In some embodiments, one or more solvent solutions of the mobile phase comprise an ion pairing agent selected from the group consisting of an imidazole, an imidazolium salt, propylamine, butylamine, pentylamine, hexylamine, hepyltamine, octylamine, trifluoroethanol, hexafluoroisopropanol, perfluoro-tert-butanol, nonafluoro-tert-butyl alcohol, trietheylammonium salt, tributylammonium salt, hexylammonium salt, dibutylammonium salt, tetrapropylammonium salt, dodecyltrimethylammonium salt, tetra(decyl)ammonium salt, dihexylammonium salt, dipropylammonium salt, myristyltrimethylammonium salt, tetraethylammonium salt, tetraheptylammonium salt, tetrahexylammonium salt, tetrakis(decyl)ammonium salt, tetramethylammonium salt, tetraoctylammonium salt, and tetrapentylammonium salt. In some embodiments, one or more solvent solutions of the mobile phase comprise imidazole, propylamine, butylamine, pentylamine, hexylamine, hepyltamine, octylamine, trifluoroethanol, hexafluoroisopropanol, perfluoro-tert-butanol, nonafluoro-tert-butyl alcohol, HAA, TBAP, TPAB, TBAC, DBAA, TEAA, DTMAC, TDAB, DHAA, DPAA MTEAB, TEAB, THepAB, THexAB, TrDAB, TMAB, TOAB, TPeABHAA, TBAP, TPAB, TBAC, DBAA, TEAA, DTMAC, or TDAB. In some embodiments, each of one or more solvents of the mobile phase comprises one ion pairing agent. In some embodiments, each of one or more solvents of the mobile phase comprises the same ion pairing agent. In some embodiments, each of one or more solvents of the mobile phase comprises an ion pairing agent selected from the group consisting of an imidazole, imidazolium salt, propylamine, butylamine, pentylamine, hexylamine, hepyltamine, octylamine, trifluoroethanol, hexafluoroisopropanol, perfluoro-tert-butanol, nonafluoro-tert-butyl alcohol, trietheylammonium salt, tributylammonium salt, hexylammonium salt, dibutylammonium salt, tetrapropylammonium salt, dodecyltrimethylammonium salt, tetra(decyl)ammonium salt, dihexylammonium salt, dipropylammonium salt, myristyltrimethylammonium salt, tetraethylammonium salt, tetraheptylammonium salt, tetrahexylammonium salt, tetrakis(decyl)ammonium salt, tetramethylammonium salt, tetraoctylammonium salt, and tetrapentylammonium salt. In some embodiments, each of one or more solvents of the mobile phase comprises imidazole, propylamine, butylamine, pentylamine, hexylamine, hepyltamine, octylamine, trifluoroethanol, hexafluoroisopropanol, perfluoro-tert-butanol, nonafluoro-tert-butyl alcohol, HAA, TBAP, TPAB, TBAC, DBAA, TEAA, DTMAC, TDAB, DHAA, DPAA MTEAB, TEAB, THepAB, THexAB, TrDAB, TMAB, TOAB, TPeABHAA, TBAP, TPAB, TBAC, DBAA, TEAA, DTMAC, or TDAB. A salt of a cation, as used herein, refers to a composition comprising the cation and an anionic counter ion. For example, a “tetrabutylammonium salt” may refer to tetrabutylammonium phosphate, tetrabutylammonium chloride, tetrabutylammonium bromide, tetrabutylammonium phosphate, or another composition comprising the cation tetrabutylammonium and an anionic counter ion. In some embodiments, the ion pairing agent comprises a cation and an anionic counter ion, wherein the cation is selected from the group consisting of imidazolium, trietheylammonium, tributylammonium, hexylammonium, dibutylammonium, tetrapropylammonium, dodecyltrimethylammonium, tetra(decyl)ammonium, dihexylammonium, dipropylammonium, myristyltrimethylammonium, tetraethylammonium, tetraheptylammonium, tetrahexylammonium, tetrakis(decyl)ammonium, tetramethylammonium, tetraoctylammonium, and tetrapentylammonium, and the anionic counter ion is selected from the group consisting of a bromide, chloride, phosphate, and acetate.

Protonated and quaternary amine ion pairing agents can be represented by the following formula:

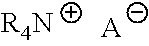

wherein each R independently is hydrogen, optionally substituted aliphatic, optionally substituted heteroaliphatic, optionally substituted aryl or optionally substituted heteroaryl; provided that at least one instance of R is not hydrogen; and A is an anionic counter ion.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups. The term “aryl” refers to a radical of a monocyclic or polycyclic (e.g., bicyclic or tricyclic) 4n+2 aromatic ring system (e.g., having 6, 10, or 14 π L electrons shared in a cyclic array) having 6-14 ring carbon atoms and zero heteroatoms provided in the aromatic ring system (“C6-14 aryl”). The term “heteroaryl” refers to a radical of a 5-14 membered monocyclic or polycyclic (e.g., bicyclic, tricyclic) 4n+2 aromatic ring system (e.g., having 6, 10, or 14 π L electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). Suitable anionic counter ions include, but are not limited to, acetate, trifluoroacetate, phosphate, chloride, bromide hexafluorophosphate, sulfate, methylsulfonate, trifluoromethylsulfonate, 1,1,1,3,3,3-hexafluoro-2-propanol (HFIP), 1,1,1,3,3,3-hexafluoro-2-methyl-2-propanol (HFMIP) and the like.

The term “optionally substituted” refers to being substituted or unsubstituted. In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction.

In some embodiments, a solvent solution of the mobile phase (e.g., a first solvent solution or a second solvent solution) comprising at least two ion pairing agents are in a molar ratio of between about 1:1,000 to about 1,000:1, such that the nucleic acids and if present, lipids, traverse the column at different rates. In some embodiments, the at least two ion pairing agents are in a molar ratio between about 1:1,000 to about 1,000:1, 1:900 to about 900:1, 1:800 to about 800:1, 1:700 to about 700:1, 1:600 to about 600:1, 1:500 to about 500:1, 1:400 to about 400:1, about 1:300 to about 300:1, about 1:200 to about 200:1, about 1:100 to about 100:1, about 50:1 to about 1:50, about 40:1 to about 1:40, about 30:1 to about 1:30, about 20:1 to about 1:20, or about 10:1 to about 1:10. In some embodiments, each solvent solution comprises at least two ion pairing agents in a molar ratio of between about 1:100 to about 100:1. In some embodiments, the at least two ion pairing agents are in a molar ratio between about 1:100 to about 100:1, 1:90 to about 90:1, 1:80 to about 80:1, 1:70 to about 70:1, 1:60 to about 60:1, 1:50 to about 50:1, 1:40 to about 40:1, about 1:30 to about 30:1, about 1:20 to about 20:1, about 1:10 to about 10:1, about 5:1 to about 1:5, about 4:1 to about 1:4, about 3:1 to about 1:3, or about 2:1 to about 1:2. In some embodiments, the at least two ion pairing agents are in a 1:1 molar ratio. In some embodiments, the at least two ion pairing agents are in a 1:10 molar ratio.

In some embodiments, a solvent solution of the mobile phase (e.g., a first solvent solution or a second solvent solution) comprises at least two ion pairing agents that are in a molar ratio of between about 1:6 to about 6:1, such that the nucleic acids and if present, lipids, traverse the column at different rates. In some embodiments, each solvent solution comprises at least two ion pairing agents in a molar ratio of between about 1:4 to about 4:1. In some embodiments, the at least two ion pairing agents are in a molar ratio between about 1:3 to about 3:1, about 1:2 to about 2:1, or about 1:1.5 to about 1.5:1. In some embodiments, the at least two ion pairing agents are in a 1:1 molar ratio.

The concentration of each ion pairing agent in a solvent solution (e.g., a first solvent solution or a second solvent solution) may range from about 0.1 mM to about 25 M (e.g., about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 2 mM, about 5 mM, about 10 mM, about 50 mM, about 100 mM, about 200 mM, about 500 mM, about 1 M, about 1.2 M, about 1.5 M, about 1.75 M, about 2M, about 2.25 M, about 2.5 M, about 2.75 M, about 3 M, about 3.25 M, about 3.5 M, about 3.75 M, about 4 M, about 4.25 M, about 4.5 M, about 4.75 M, about 5 M, about 5.5 M, about 6 M, about 6.5 M, about 7 M, about 7.5 M, about 8 M, about 8.5 M, about 9 M, about 9.5 M, about 10 M, about 11 M, about 12 M, about 13 M, about 14 M, about 15 M, about 16 M, about 17 M, about 18 M, about 19 M, or about 20 M), inclusive. In some embodiments, the concentration of an ion pairing agent in a mobile phase (e.g., a first solvent solution or a second solvent solution) ranges from about 0.1 mM-100 mM, about 10 mM-20 M, 20 mM-15 M, 30 mM-12 M, 40 mM-10 M, 50 mM-8 M, 75 mM-5 M, 100 mM-2.5 M, 125 mM-2 M, 150 mM-1.5 M, 175 mM-1 M, or 200 mM-500 mM. In some embodiments, the concentration of each of the ion pairing agents independently ranges from about 0.1 mM-100 mM, about 10 mM-20 M, 20 mM-15 M, 30 mM-12 M, 40 mM-10 M, 50 mM-8 M, 75 mM-5 M, 100 mM-2.5M, 125 mM-2M, 150 mM-1.5M, 175 mM-1 M, or 200 mM-500 mM. In some embodiments, a first or second solvent solution comprises a single ion pairing agent, which is present in an amount from about 0.1 mM-100 mM, about 10 mM-20M, 20 mM-15M, 30 mM-12M, 40 mM-10M, 50 mM-8M, 75 mM-5M, 100 mM-2.5 M, 125 mM-2 M, 150 mM-1.5 M, 175 mM-1 M, or 200 mM-500 mM.

The concentration of each ion pairing agent in a solvent solution (e.g., a first solvent solution or a second solvent solution) may range from about 0.1 mM to about 2 M (e.g., about 0.1 mM, about 0.2 mM, about 0.3 mM, about 0.4 mM, about 0.5 mM, about 0.6 mM, about 0.7 mM, about 0.8 mM, about 0.9 mM, about 2 mM, about 5 mM, about 10 mM, about 50 mM, about 100 mM, about 200 mM, about 500 mM, about 1 M, about 1.2 M, about 1.5 M, or about 2M), inclusive. In some embodiments, the concentration of an ion pairing agent in a mobile phase (e.g., a first solvent solution or a second solvent solution) ranges from about 0.1 mM-100 mM, about 10 mM-1M, 40 mM-300 mM, 50 mM-500 mM, 75 mM-400 mM, 100 mM-300 mM, 200-300 mM, 200-250 mM, or 250-300 mM. In some embodiments, the concentration of each of the ion pairing agents independently ranges from about 0.1 mM-100 mM, about 10 mM-1M, 40 mM-300 mM, 50 mM-500 mM, 75 mM-400 mM, 100 mM-300 mM, 200-300 mM, 200-250 mM, or 250-300 mM. In some embodiments, two ion pairing agents are present at concentrations of about 0.1 mM:1 mM, 0.2 mM:2 mM, 0.3 mM:3 mM, 0.4 mM:4 mM, 0.5 mM:5 mM, 0.6 mM:6 mM, 0.7 mM:7 mM, 0.8 mM:8 mM, 0.9 mM:9 mM, 1 mM:10 mM, 1 mM: 1 mM, 2 mM:2 mM, 3 mM:3 mM, 4 mM:4 mM, 5 mM:5 mM, 6 mM:6 mM, 7 mM:7 mM, 8 mM:8 mM, 9 mM:9 mM, 10 mM:10 mM, 20 mM:40 mM, 50 mM:50 mM, 50 mM:60 mM, 50 mM:75 mM, 50 mM:100 mM, 50 mM:150 mM, 100 mM:100 mM, 100 mM:125 mM, 100 mM:150 mM, 100 mM:175 mM, 100 mM:200 mM, 100 mM:200 mM, 100 mM:250 mM, 100 mM:300 mM, 125 mM:125 mM, 125 mM:150 mM, 125 mM:175 mM, 125 mM: 200 mM, 125 mM:250 mM, 125 mM:300 mM, 150 mM:175 mM, 150 mM:200 mM, 150 mM: 250 mM, 150 mM:300 mM, 200 mM:200 mM, 200 mM:250 mM, 200 mM:300 mM, 250 mM:250 mM, 250 mM:300 mM, or 300 mM:300 mM.

Ion pairing agents are generally dispersed within a mobile phase. As used herein, a “mobile phase” is an aqueous solution comprising water and/or one or more organic solvents used to carry an HPLC analyte (or analytes), such as a nucleic acid, mixture of nucleic acids, or a pharmaceutical composition comprising a nucleic acid or mixture of nucleic acids, through an HPLC column. In some embodiments, a mobile phase for use in HPLC methods as described by the disclosure is comprised of multiple (e.g., 2, 3, 4, 5, or more) solvent solutions. In some embodiments of the HPLC methods described by the disclosure, the mobile phase comprises two solvent solutions, a first solvent solution and a second solvent solution (e.g., Mobile Phase A, and Mobile Phase B). In some embodiments, a solvent solution comprises at least two ion pairing agents in a molar ratio of 1:1,000 to 1,000:1. In some embodiments, each solvent solution (e.g., the first solvent solution and the second solvent solution) comprises at least two ion pairing agents in a molar ratio of 1:1,000 to 1,000:1. In some embodiments, a solvent solution comprises at least two ion pairing agents in a molar ratio of 1:100 to 100:1. In some embodiments, each solvent solution (e.g., the first solvent solution and the second solvent solution) comprises at least two ion pairing agents in a molar ratio of 1:100 to 100:1. In some embodiments, a solvent solution comprises at least two ion pairing agents in a molar ratio of 1:75 to 75:1. In some embodiments, each solvent solution (e.g., the first solvent solution and the second solvent solution) comprises at least two ion pairing agents in a molar ratio of 1:75 to 75:1. In some embodiments, a solvent solution comprises at least two ion pairing agents in a molar ratio of 1:50 to 50:1. In some embodiments, each solvent solution (e.g., the first solvent solution and the second solvent solution) comprises at least two ion pairing agents in a molar ratio of 1:50 to 50:1. In some embodiments, a solvent solution comprises at least two ion pairing agents in a molar ratio of 1:25 to 25:1. In some embodiments, each solvent solution (e.g., the first solvent solution and the second solvent solution) comprises at least two ion pairing agents in a molar ratio of 1:25 to 25:1. In some embodiments, a solvent solution comprises at least two ion pairing agents in a molar ratio of 1:10 to 10:1. In some embodiments, each solvent solution (e.g., the first solvent solution and the second solvent solution) comprises at least two ion pairing agents in a molar ratio of 1:10 to 10:1. In some embodiments, a solvent solution comprises at least two ion pairing agents in a molar ratio of 1:6 to 6:1. In some embodiments, each solvent solution (e.g., the first solvent solution and the second solvent solution) comprises at least two ion pairing agents in a molar ratio of 1:6 to 6:1. In some embodiments, a solvent solution comprises at least two ion pairing agents in a molar ratio of 1:4 to 4:1. In some embodiments, each solvent solution (e.g., the first solvent solution and the second solvent solution) comprises at least two ion pairing agents in a molar ratio of 1:4 to 4:1.

In some embodiments, one or more solvent solutions (e.g., 1, 2, 3, 4, 5, or more) of the mobile phase comprise one or more volatile salts (e.g., 2, 3, 4, 5, or more). As used herein, a “volatile salt” refers to a salt that is capable of being evaporated with a liquid of the mobile phase. In the liquid mobile phase, volatile salts associate with nucleic acids of a mixture, and ionization of the volatile salt in the eluted mRNA promotes ionization of the mRNA. Contaminants such as other ions and small molecules are more efficiently ionized than nucleic acids, and noise from the contaminants can therefore obscure the signal of mRNAs in mass spectrometry. However, inclusion of a volatile salt promotes ionization of the mRNA, allowing mRNA to be detected in mass spectrometry, thereby increasing the signal-to-noise ratio. Therefore, volatile salts improve the resolution of mass spectrometry-based methods of analyzing mRNAs purified by HILIC-based methods. In some embodiments, one or more volatile salts utilized in the methods described by the disclosure is a cationic salt. In some embodiments, the at least one volatile salt in the first and/or second solvent solution is selected from the group consisting of formic acid, acetic acid, trifluoroacetic acid, ammonium formate, ammonium acetate, ammonium hydroxide, triethylamine acetate, triethylamine formate, diethylamine acetate, diethylamine formate, piperidine acetate, piperidine formate, ammonium bicarbonate, borate, hydride, 4-methylmorpholine, 1-methylpiperidine, pyrrolidine acetate, and pyrrolidine formate. In some embodiments, the volatile salt is formic acid. In some embodiments, the formic acid is present at a concentration of about 1 mM, 10 mM, 20 mM, 30 mM, 40 mM, 50 mM, 60 mM, 70 mM, 80 mM, 90 mM, or 100 mM. In some embodiments, the volatile salt is acetic acid. In some embodiments, the acetic acid is present at a concentration of about 1 mM, 10 mM, 20 mM, 30 mM, 40 mM, 50 mM, 60 mM, 70 mM, 80 mM, 90 mM, or 100 mM. In some embodiments, the volatile salt is trifluoroacetic acid. In some embodiments, the trifluoroacetic acid is present at a concentration of about 1 mM, 10 mM, 20 mM, 30 mM, 40 mM, 50 mM, 60 mM, 70 mM, 80 mM, 90 mM, or 100 mM. In some embodiments, the volatile salt is ammonium formate. In some embodiments, the ammonium formate is present at a concentration of about 1 mM, 10 mM, 20 mM, 30 mM, 40 mM, 50 mM, 60 mM, 70 mM, 80 mM, 90 mM, or 100 mM. In some embodiments, the volatile salt is ammonium acetate. In some embodiments, the ammonium acetate is present at a concentration of about 1 mM, 10 mM, 20 mM, 30 mM, 40 mM, 50 mM, 60 mM, 70 mM, 80 mM, 90 mM, or 100 mM. In some embodiments, the volatile salt is diethylamine formate. In some embodiments, the diethylamine formate is present at a concentration of about 1 mM, 10 mM, 20 mM, 30 mM, 40 mM, 50 mM, 60 mM, 70 mM, 80 mM, 90 mM, or 100 mM. In some embodiments, the volatile salt is piperidine acetate. In some embodiments, the piperidine acetate is present at a concentration of about 1 mM, 10 mM, 20 mM, 30 mM, 40 mM, 50 mM, 60 mM, 70 mM, 80 mM, 90 mM, or 100 mM. In some embodiments, the volatile salt is piperidine formate. In some embodiments, the piperidine formate is present at a concentration of about 1 mM, 10 mM, 20 mM, 30 mM, 40 mM, 50 mM, 60 mM, 70 mM, 80 mM, 90 mM, or 100 mM. In some embodiments, the volatile salt is ammonium bicarbonate. In some embodiments, the ammonium bicarbonate is present at a concentration of about 1 mM, 10 mM, 20 mM, 30 mM, 40 mM, 50 mM, 60 mM, 70 mM, 80 mM, 90 mM, or 100 mM. In some embodiments, the volatile salt is borate. In some embodiments, the borate is present at a concentration of about 1 mM, 10 mM, 20 mM, 30 mM, 40 mM, 50 mM, 60 mM, 70 mM, 80 mM, 90 mM, or 100 mM. In some embodiments, the volatile salt is hydride. In some embodiments, the hydride is present at a concentration of about 1 mM, 10 mM, 20 mM, 30 mM, 40 mM, 50 mM, 60 mM, 70 mM, 80 mM, 90 mM, or 100 mM. In some embodiments, the volatile salt is 4-methylmorpholine. In some embodiments, the 4-methylmorpholine is present at a concentration of about 1 mM, 10 mM, 20 mM, 30 mM, 40 mM, 50 mM, 60 mM, 70 mM, 80 mM, 90 mM, or 100 mM. In some embodiments, the volatile salt is 1-methylpiperidine. In some embodiments, the 1-methylpiperidine is present at a concentration of about 1 mM, 10 mM, 20 mM, 30 mM, 40 mM, 50 mM, 60 mM, 70 mM, 80 mM, 90 mM, or 100 mM. In some embodiments, the volatile salt is pyrrolidine acetate. In some embodiments, the pyrrolidine acetate is present at a concentration of about 1 mM, 10 mM, 20 mM, 30 mM, 40 mM, 50 mM, 60 mM, 70 mM, 80 mM, 90 mM, or 100 mM. In some embodiments, the volatile salt is pyrrolidine formate. In some embodiments, the pyrrolidine formate is present at a concentration of about 1 mM, 10 mM, 20 mM, 30 mM, 40 mM, 50 mM, 60 mM, 70 mM, 80 mM, 90 mM, or 100 mM.

The concentration of each volatile salt in a solvent solution (e.g., a first solvent solution or a second solvent solution) may range from about 1 mM to about 25 M (e.g., about 1 mM, about 2 mM, about 3 mM, about 4 mM, about 5 mM, about 6 mM, about 7 mM, about 8 mM, about 9 mM, about 10 mM, about 20 mM, about 30 mM, about 40 mM, about 50 mM, about 100 mM, about 200 mM, about 500 mM, about 1 M, about 1.2 M, about 1.5 M, about 1.75 M, about 2M, about 2.25 M, about 2.5 M, about 2.75 M, about 3 M, about 3.25 M, about 3.5 M, about 3.75 M, about 4 M, about 4.25 M, about 4.5 M, about 4.75 M, about 5 M, about 5.5 M, about 6 M, about 6.5 M, about 7 M, about 7.5 M, about 8 M, about 8.5 M, about 9 M, about 9.5 M, about 10 M, about 11 M, about 12 M, about 13 M, about 14 M, about 15 M, about 16 M, about 17 M, about 18 M, about 19 M, or about 20 M), inclusive. In some embodiments, the concentration of a volatile salt in a mobile phase (e.g., a first solvent solution or a second solvent solution) ranges from about 0.1 mM-200 mM, 1 mM-2 M, 2 mM-1.5 M, 3 mM-1.2 M, 4 mM-1 M, 5 mM-800 mM, 7.5 mM-500 mM, 10 mM-250 mM, 12.5 mM-200 mM, 15 mM-150 mM, 17.5 mM-100 mM, or 20 mM-500 mM. In some embodiments, the concentration of each volatile salt independently ranges from about 0.1 mM-200 mM, 1 mM-2 M, 2 mM-1.5 M, 3 mM-1.2 M, 4 mM-1 M, 5 mM-800 mM, 7.5 mM-500 mM, 10 mM-250 mM, 12.5 mM-200 mM, 15 mM-150 mM, 17.5 mM-100 mM, or 20 mM-500 mM. In some embodiments, a first or second solvent solution comprises a single volatile salt, which is present in an amount from about 0.1 mM-200 mM, 1 mM-2 M, 2 mM-1.5 M, 3 mM-1.2 M, 4 mM-1 M, 5 mM-800 mM, 7.5 mM-500 mM, 10 mM-250 mM, 12.5 mM-200 mM, 15 mM-150 mM, 17.5 mM-100 mM, or 20 mM-500 mM.

In some embodiments, at least one solvent solution of the mobile phase comprises an organic solvent. Generally, an HPLC mobile phase comprises a polar organic solvent. Examples of polar organic solvents suitable for inclusion in a mobile phase include but are not limited to alcohols, ketones, nitrates, esters, amides and alkylsulfoxides. In some embodiments, the mobile phase (e.g., at least one solvent solution of the mobile phase) comprises one or more organic solvents selected from the group consisting of polar aprotic solvents, C₁₋₄ alkanols, C₁₋₆ alkanediols, and C₂₋₄ alkanoic acids. In some embodiments, the mobile phase (e.g., at least one solvent solution of the mobile phase) comprises one or more organic solvents selected form the group consisting of acetone, acetonitrile, dimethylformamide, dimethylsulfoxide (DMSO), ethanol, hexylene glycol, isopropanol, methanol, methyl acetate, propanol, and tetrahydrofuran. In some embodiments, the mobile phase (e.g., at least one solvent solution of the mobile phase) comprises acetonitrile. In some embodiments, a mobile phase (e.g., at least one solvent solution of the mobile phase) comprises additional components, for example as described in U.S. Patent Publication US 2005/0011836, the entire contents of which are incorporated herein by reference.

The concentration of organic solvent in a mobile phase (e.g., each solvent solution of the mobile phase) can vary. For example, in some embodiments, the volume percentage (v/v) of an organic solvent in a mobile phase varies from 0% (absent) to about 100% of a mobile phase. In some embodiments, the volume percentage of organic solvent in a mobile phase (e.g., at least one solvent solution of the mobile phase) is between about 5% and about 75% v/v. In some embodiments, the volume percentage of organic solvent in a mobile phase (e.g., at least one solvent solution of the mobile phase) is between about 25% and about 60% v/v. In some embodiments, the volume percentage of organic solvent in a mobile phase (e.g., at least one solvent solution of the mobile phase) is at least about 50% v/v. In some embodiments, the volume percentage of organic solvent in a mobile phase (e.g., at least one solvent solution of the mobile phase) is about 50% to about 95%, about 55% to about 90%, about 60% to about 85%, about 65% to about 80%, or about 70% v/v to about 75% v/v. In some embodiments, the concentration of organic solvent in a mobile phase (e.g., at least one solvent solution of the mobile phase) is about 5%, about 10%, about 15%, about 20%, about 25%, about 30%, about 35%, about 40%, about 45%, about 50%, about 55%, about 60%, about 65%, about 70%, about 75%, about 80%, about 85%, about 90% v/v, or about 95% v/v.

In some embodiments, the first solvent solution does not comprise an organic solvent. In some embodiments, the volume percentage of organic solvent in the second solvent solution is at least about 50% v/v. In some embodiments, the volume percentage of organic solvent in the second solvent solution is about 50% to about 95%, about 55% to about 90%, about 60% to about 85%, about 65% to about 80%, or about 70% v/v to about 75% v/v. In some embodiments, the volume percentage of organic solvent in the second solvent solution is about 50%, about 55%, about 60%, about 65%, about 70%, about 75%, about 80%, about 85%, about 90% v/v, or about 95% v/v.

The concentration of two or more solvent solutions in a mobile phase can vary. For example, in a mobile phase comprising two solvent solutions (e.g., a first solvent solution and a second solvent solution), the volume percentage of the first solvent solution may range from about 0% (absent) to about 100%. In some embodiments, the volume percentage of the first solvent solution may range from about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% v/v.

Conversely, in some embodiments, the volume percentage of the second solvent solution of a mobile phase may range from about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% v/v.

In some embodiments, the ratio of the first solvent solution to the second solvent solution is held constant (e.g., isocratic) during elution of the nucleic acid. However, the skilled artisan will appreciate that in other embodiments, the relative ratio of the first solvent solution to the second solvent solution can vary throughout the elution step. For example, in some embodiments, the ratio of the first solvent solution is increased relative to the second solvent solution during the elution step. In some embodiments, the ratio of the first solvent solution is decreased relative to the second solvent solution during the elution step.

The concentration of one or more ion pairing agents in a mobile phase (e.g., a solvent solution) can vary. The relative ratios of the at least two ion pairing agents in a mobile phase (or solvent solution) may vary or be held constant (e.g., isocratic) during the eluting step. In some embodiments, the ratio of a first ion pairing agent is increased relative to a second ion pairing agent during the elution step. In some embodiments, the ratio of a first ion pairing agent is increased relative to a second ion pairing agent during the elution step. For example, in some embodiments, the ratio of the first ion pairing agent to the second ion pairing agent ranges from about 4:1 to about 1:4, about 3:1 to about 1:3, about 2:1 to about 1:2, or about 1:1 to 1:3.

The mobile phase (e.g., a solvent solution) may be gradient or isocratic with respect to the concentration of one or more organic solvents.

In some embodiments, the mobile phase has a pH of about 6.5 to about 10. In some embodiments, the mobile phase has a pH of about 6.5 to about 7, about 7 to about 7.5, about 7.5 to about 8.0, about 8.0 to about 8.5, about 8.5 to about 9.0, about 9.0 to about 9.5, or about 9.5 to about 10. In some embodiments, the mobile phase has a pH of 6.5. In some embodiments, the mobile phase has a pH of 6.5 or greater. In some embodiments, the mobile phase has a pH of 6.6. In some embodiments, the mobile phase has a pH of 6.6 or greater. In some embodiments, the mobile phase has a pH of 6.7. In some embodiments, the mobile phase has a pH of 6.7 or greater. In some embodiments, the mobile phase has a pH of 6.8. In some embodiments, the mobile phase has a pH of 6.8 or greater. In some embodiments, the mobile phase has a pH of 6.9. In some embodiments, the mobile phase has a pH of 6.9 or greater. In some embodiments, the mobile phase has a pH of 7.0. In some embodiments, the mobile phase has a pH of 7.0 or greater. In some embodiments, the mobile phase has a pH of 7.1. In some embodiments, the mobile phase has a pH of 7.1 or greater. In some embodiments, the mobile phase has a pH of 7.2. In some embodiments, the mobile phase has a pH of 7.2 or greater. In some embodiments, the mobile phase has a pH of 7.3. In some embodiments, the mobile phase has a pH of 7.3 or greater. In some embodiments, the mobile phase has a pH of 7.4. In some embodiments, the mobile phase has a pH of 7.4 or greater. In some embodiments, the mobile phase has a pH of 7.5. In some embodiments, the mobile phase has a pH of 7.5 or greater. In some embodiments, the mobile phase has a pH of 7.6. In some embodiments, the mobile phase has a pH of 7.6 or greater. In some embodiments, the mobile phase has a pH of 7.7. In some embodiments, the mobile phase has a pH of 7.7 or greater. In some embodiments, the mobile phase has a pH of 7.8. In some embodiments, the mobile phase has a pH of 7.8 or greater. In some embodiments, the mobile phase has a pH of 7.9. In some embodiments, the mobile phase has a pH of 7.9 or greater. In some embodiments, the mobile phase has a pH of 8.0. In some embodiments, the mobile phase has a pH of 8.0 or greater. In some embodiments, the mobile phase has a pH of 8.1. In some embodiments, the mobile phase has a pH of 8.1 or greater. In some embodiments, the mobile phase has a pH of 8.2. In some embodiments, the mobile phase has a pH of 8.2 or greater. In some embodiments, the mobile phase has a pH of 8.3. In some embodiments, the mobile phase has a pH of 8.3 or greater. In some embodiments, the mobile phase has a pH of 8.4. In some embodiments, the mobile phase has a pH of 8.4 or greater. In some embodiments, the mobile phase has a pH of 8.5. In some embodiments, the mobile phase has a pH of 8.5 or greater. In some embodiments, the mobile phase has a pH of 8.6. In some embodiments, the mobile phase has a pH of 8.6 or greater. In some embodiments, the mobile phase has a pH of 8.7. In some embodiments, the mobile phase has a pH of 8.7 or greater. In some embodiments, the mobile phase has a pH of 8.8. In some embodiments, the mobile phase has a pH of 8.8 or greater. In some embodiments, the mobile phase has a pH of 8.9. In some embodiments, the mobile phase has a pH of 8.9 or greater. In some embodiments, the mobile phase has a pH of 9.0. In some embodiments, the mobile phase has a pH of 9.0 or greater. In some embodiments, the mobile phase has a pH of 9.1. In some embodiments, the mobile phase has a pH of 9.1 or greater. In some embodiments, the mobile phase has a pH of 9.2. In some embodiments, the mobile phase has a pH of 9.2 or greater. In some embodiments, the mobile phase has a pH of 9.3. In some embodiments, the mobile phase has a pH of 9.3 or greater. In some embodiments, the mobile phase has a pH of 9.4. In some embodiments, the mobile phase has a pH of 9.4 or greater. In some embodiments, the mobile phase has a pH of 9.5. In some embodiments, the mobile phase has a pH of 9.5 or greater. In some embodiments, the mobile phase has a pH of 9.6. In some embodiments, the mobile phase has a pH of 9.6 or greater. In some embodiments, the mobile phase has a pH of 9.7. In some embodiments, the mobile phase has a pH of 9.7 or greater. In some embodiments, the mobile phase has a pH of 9.8. In some embodiments, the mobile phase has a pH of 9.8 or greater. In some embodiments, the mobile phase has a pH of 9.9. In some embodiments, the mobile phase has a pH of 9.9 or greater. In some embodiments, the mobile phase has a pH of 10. In some embodiments, the mobile phase has a basic pH.

Any suitable column (e.g., stationary phase) may be used in the methods described by the disclosure. Generally, a “HPLC column” is a solid structure or support that contains a medium (e.g. a stationary phase) through which the mobile phase and HPLC sample (e.g., a sample containing HPLC analytes, such as nucleic acids) is eluted. Without wishing to be bound by any particular theory, the composition and chemical properties of the stationary phase determine the retention time of HPLC analytes. In some embodiments of HPLC methods described by the disclosure, the stationary phase is non-polar. Examples of non-polar stationary phases include but are not limited to resin, silica (e.g., alkylated and non-alkylated silica), polystyrenes (e.g., alkylated and non-alkylated polystyrenes), polystyrene divinylbenzenes, etc. In some embodiments, a stationary phase comprises particles, for example porous particles. In some embodiments, a stationary phase (e.g., particles of a stationary phase) is hydrophobic (e.g., made of an intrinsically hydrophobic material, such as polystyrene divinylbenzene), or comprise hydrophobic functional groups. In some embodiments, a stationary phase is a membrane or monolithic stationary phase.

The particle size (e.g., as measured by the diameter of the particle) of an HPLC stationary phase can vary. In some embodiments, the particle size of a HPLC stationary phase ranges from about 1 μm to about 100 μm (e.g., any value between 1 and 100, inclusive) in diameter. In some embodiments, the particle size of a HPLC stationary phase ranges from about 2 μm to about 10 μm, about 2 μm to about 6 μm, or about 4 μm in diameter. The pore size of particles (e.g., as measured by the diameter of the pore) can also vary. In some embodiments, the particles comprise pores having a diameter of about 100 Å to about 10,000 Å. In some embodiments, the particles comprise pores having a diameter of about 100 Å to about 5,000 Å, about 100 Å to about 1,000, or about 1,000 to about 2,000. In some embodiments, the stationary phase comprises polystyrene divinylbenzene, for example as used in PLRP-S 4000 columns or DNAPac-RP columns.

In some embodiments, the stationary phase is comprised in a column selected from the group consisting of an AdvanceBio column, Agilent column, Agilent Bio column, Agilent Prep column, ChiraDex column, ChromoSpher column, HC/TC column, Hi-Plex column, Hypersil column, InfinityLab column, IonoSpher column, LiChroPrep column, LiChrosorb column, LiChrospher column, MetaCarb column, MetaChem column, MetaSil column, Microsorb column, MicroSpher column, Monochrom column, Monolith Bio column, OmniSpher column, PetroSpher column, PL column, Plaquagel-OH column, PIgel column, PLRP-S column, PlusPore column, Polaris column, Poroshell column, ProSEC column, PRP column, Purospher column, Pursuit column, Pursuit XRs column, SepTech column, Sumichiral column, Superspher column, Ultron column, VariTide column, Zorbax column, Zorbax Eclipse column, Zorbax Eclipse Plus column, Zorbax RRHD column, Zorbax RRHD Bonus-RP column, Zorbax RRHD Eclipse column, Zorbax RRHD Eclipse Plus column, or Zorbax RRHD SB column. In some embodiments, the column is an AdvanceBio column. In some embodiments, the column is an Agilent column. In some embodiments, the column is an Agilent Bio column. In some embodiments, the column is an Agilent Prep column. In some embodiments, the column is a ChiraDex column. In some embodiments, the column is a ChromoSpher column. In some embodiments, the column is a HC/TC column. In some embodiments, the column is a Hi-Plex column. In some embodiments, the column is a Hypersil column. In some embodiments, the column is a InfinityLab column. In some embodiments, the column is a IonoSpher column. In some embodiments, the column is a LiChroPrep column. In some embodiments, the column is a LiChrosorb column. In some embodiments, the column is a LiChrospher column. In some embodiments, the column is a MetaCarb column. In some embodiments, the column is a MetaChem column. In some embodiments, the column is a MetaSil column. In some embodiments, the column is a Microsorb column. In some embodiments, the column is a MicroSpher column. In some embodiments, the column is a Monochrom column. In some embodiments, the column is a Monolith Bio column. In some embodiments, the column is a OmniSpher column. In some embodiments, the column is a PetroSpher column. In some embodiments, the column is a PL column. In some embodiments, the column is a Plaquagel-OH column. In some embodiments, the column is a PIgel column. In some embodiments, the column is a PLRP-S column. In some embodiments, the column is a PlusPore column. In some embodiments, the column is a Polaris column. In some embodiments, the column is a Poroshell column. In some embodiments, the column is a ProSEC column. In some embodiments, the column is a PRP column. In some embodiments, the column is a Purospher column. In some embodiments, the column is a Pursuit column. In some embodiments, the column is a Pursuit XRs column. In some embodiments, the column is a SepTech column. In some embodiments, the column is a Sumichiral column. In some embodiments, the column is a Superspher column. In some embodiments, the column is an Ultron column. In some embodiments, the column is a VariTide column. In some embodiments, the column is a Zorbax column. In some embodiments, the column is a Zorbax Eclipse column. In some embodiments, the column is a Zorbax Eclipse Plus column. In some embodiments, the column is a Zorbax RRHD column. In some embodiments, the column is a Zorbax RRHD Bonus-RP column. In some embodiments, the column is a Zorbax RRHD Eclipse column. In some embodiments, the column is a Zorbax RRHD Eclipse Plus column. In some embodiments, the column is a Zorbax RRHD SB column.

In some embodiments, the injection volumes of the sample (e.g., a pharmaceutical preparation) range from about 10 μL to about 100 μL, about 10 μL to about 50 μL, about 20 μL to about 50 μL, about 20 μL to about 70 μL, or about 50 μL to about 100 μL.

The methods of the disclosure allow the use of small doses of pharmaceutical preparations. Accordingly, dose preparation amounts of a pharmaceutical composition may be tested using the methods of the disclosure. In some embodiments, a target nucleic acid is present in the pharmaceutical composition (e.g., lipid-based pharmaceutical composition) in an amount ranging from about 0.05 mg/mL to about 1 mg/mL (e.g., 0.05, 006, 0.07, 0.08, 0.09, 0.1, 0.2. 0.3. 0.4, 0.5, 0.6, 0.7, 0.8, 0.9, or 1 mg/mL). In some embodiments, the target nucleic acid is present in the pharmaceutical composition at about 0.1 mg/mL.

The temperature of the column (e.g., the stationary phase within the column) can vary. In some embodiments, the column has a temperature from about 20° C. to about 99° C. (e.g., any temperature between 20° C. and 99° C. In some embodiments, the column has a temperature from about 20° C. to about 60° C. (e.g., any temperature between 20° C. and 60° C., for example 15 about 20° C., about 30° C., about 40° C., about 50° C., or about 60° C.). In some embodiments, the column has a temperature of about 20° C. In some embodiments, the column has a temperature of about 30° C. In some embodiments, the column has a temperature of about 40° C. In some embodiments, the column has a temperature of about 50° C. In some embodiments, the column has a temperature of about 60° C.

In some embodiments, the HILIC-based methods provided herein allow purification of nucleic acids (e.g., mRNAs) having lengths greater than 200, greater than 300, greater than 400, greater than 500, greater than 600, greater than 700, greater than 800, greater than 900, or greater than 1000 nucleotides. In some embodiments, the one or more nucleic acids of the composition added to the stationary phase has a length between 300-500 nucleotides, 500-1000 nucleotides, 1000-1500 nucleotides, 1500-2000 nucleotides, 2000-2500 nucleotides, 2500-3000 nucleotides, 3000-3500 nucleotides, 3500-4000 nucleotides, 4000-4500 nucleotides, or 4500-5000 nucleotides. In some embodiments, the HILIC-based methods provided herein allow purification of nucleic acids (e.g., mRNAs) having lengths greater than 200, greater than 300, greater than 400, greater than 500, greater than 600, greater than 700, greater than 800, greater than 900, greater than 1000 nucleotides, greater than 1100 nucleotides, greater than 1200 nucleotides, greater than 1300 nucleotides, greater than 1400 nucleotides, greater than 1500 nucleotides, grater than 1600 nucleotides, greater than 1700 nucleotides, greater than 1800 nucleotides, greater than 1900 nucleotides, or greater than 2000 nucleotides.

In some embodiments, the mRNA added to the stationary phase does not comprise a poly(A) tail. In some embodiments, a poly(A) tail has been removed from the mRNA (e.g., by RNase H cleavage) before the mRNA is added to the stationary phase of the column. RNase H cleaves RNA in a double-stranded DNA:RNA hybrid, such as an mRNA that is bound to a DNA oligonucleotide. Binding of a DNA oligonucleotide to a region of the mRNA upstream from the poly(A) tail of an mRNA, such as a sequence within the 3′ UTR, allows RNase H to cleave the mRNA into two fragments, with one cleavage fragment containing the poly(A) tail and a terminal portion of the 3′ UTR, and the other cleavage fragment containing the rest of the mRNA. Because mRNAs having the same cap, 5′ UTR, open reading frame, and 3′ UTR may vary in the lengths of their poly(A) tails, and thus have different masses, RNase H-mediated cleavage of the poly(A) tail corrects the mass differences that arise from different poly(A) tail lengths, thereby producing a homogenous population of mRNAs and improving the resolution of mass spectrometry.

### Mass Spectrometry

Aspects of the present disclosure relate to methods of analyzing nucleic acids (e.g., mRNAs) by mass spectrometry. Mass spectrometry determines the mass-to-charge ratios of analytes (e.g., nucleic acids) in a composition by ionizing the analytes, accelerating them through a magnetic field, which deflects the ions based on their size, with lighter and more strongly charged ions being deflected more strongly. Longer nucleic acids contain more sites that can be deprotonated, and more deprotonated sites impart a higher charge state to the nucleic acid. These higher charge states cause merging of individual mass-to-charge peaks in spectra generated by mass spectrometry, which reduces the ability of mass spectrometry to resolve the mass of larger nucleic acids. Additionally, when one nucleic acid species is differentially deprotonated, the average abundance of each form of the nucleic acid with a given charge state is reduced, as is the signal corresponding to a given mass-to-charge position on a mass-to-charge spectrum. Noise from impurities can thus more easily obscure the mass-to-charge signal from nucleic acids. Surprisingly, purifying nucleic acids by hydrophilic interaction chromatography reduced the average charge state of the purified nucleic acids, improving the signal-to-noise ratio and peak resolution spectra generated by mass spectrometry.

Purified nucleic acids may be ionized and analyzed by any of multiple mass spectrometry methods known in the art, including matrix-assisted laser desorption/ionization (MALDI), infrared MALDI (IR-MALDI), electrospray ionization (ESI), time-of-flight (TOF) mass, spectrometry, and combinations thereof. In some embodiments, the method comprises analyzing the purified nucleic acid by ESI-TOF, MALDI-TOF, or Q-TOF mass spectrometry.

In some embodiments, the method uses matrix-assisted laser desorption/ionization (MALDI) to generate ionized nucleic acids for analysis by mass spectrometry. MALDI involves laser pulses focused on a small sample plate comprising analyte molecules (nucleic acids) embedded in either a solid or liquid matrix comprising a small, highly absorbing compound. The laser pulses transfer energy to the matrix causing a microscopic ablation and concomitant ionization of the analyte molecules, producing a gaseous plume of intact, charged nucleic acids.

In some embodiments, the method uses electrospray ionization (ESI) mass spectrometry to generate ionized nucleic acids for analysis by mass spectrometry. ESI mass spectrometry produces ions for analysis by applying a high voltage to a liquid to create an aerosol. Aerosol formation produces a suspension of small liquid particles, each containing ions to be analyzed.

In some embodiments, the method uses time-of-flight (TOF) mass spectrometry to analyze ionized nucleic acids. In TOF mass spectrometry, the ions generated are accelerated to a fixed kinetic energy by a strong electric field and then pass through an electric field-free region in vacuum in which the ions travel with a velocity corresponding to their respective mass-to-charge ratios (m/z). The smaller m/z ions will travel through the vacuum region faster than the larger m/z ions thereby causing a separation. At the end of the electric field-free region, the ions collide with a detector that generates a signal as each set of ions of a particular mass-to-charge ratio strikes the detector. Usually for a given assay, 10 to 100 mass spectra resulting from individual laser pulses are summed together to make a single composite mass spectrum with an improved signal-to-noise ratio. The mass of an ion (such as a charged nucleic acid) is measured by using its velocity to determine the mass-to-charge ratio by time-of-flight analysis. In other words, the mass of the molecule directly correlates with the time it takes to travel from the sample plate to the detector.

Some embodiments of the methods provided herein use quadrupole TOF (Q-TOF) mass spectrometry to analyze the mass of ionized nucleic acids (e.g., mRNAs). Q-TOF utilizes a quadrupole ion trap, which exposes ions to two pairs of opposing magnetic fields, each pair emitting a magnetic fields that are orthogonal to the magnetic fields emitted by the other pair, to trap ions in the center of the four magnetic fields. Trapped ions can then be propelled in a direction perpendicular to the direction of all four magnetic fields, for passage through a TOF tube and time-of-flight analysis as described above. See, e.g., March. J Mass Spectrom. 1997. 32:351-369.

In some embodiments, the TOF mass spectrometry comprises directing ionized nucleic acids through a TOF tube having a length between about 1-5 m, 1-4 m, 1-3 m, 1-2 m, 2-5 m, or 2-4 m, inclusive.

In some embodiments, the mass spectrometry is conducted using a Q-TOF mass spectrometer. In some embodiments, the mass spectrometer is an Agilent 6530 mass spectrometer. In some embodiments, the mass spectrometer is an Agilent 6545 mass spectrometer. In some embodiments, the mass spectrometer is an Agilent 6545XT mass spectrometer. In some embodiments, the mass spectrometer is an Agilent 6546 mass spectrometer. In some embodiments, the mass spectrometer is an Agilent 6550 mass spectrometer. In some embodiments, the mass spectrometer is an Agilent 6560 mass spectrometer. In some embodiments, the mass spectrometer is an Agilent 7200 mass spectrometer. In some embodiments, the mass spectrometer is an Agilent 7200B mass spectrometer. In some embodiments, the mass spectrometer is an Agilent 7250 mass spectrometer. In some embodiments, the mass spectrometer is a Bruker compact ESI mass spectrometer. In some embodiments, the mass spectrometer is a Bruker impact II ESI mass spectrometer. In some embodiments, the mass spectrometer is a Bruker maXis II mass spectrometer. In some embodiments, the mass spectrometer is a SCIEX X500B mass spectrometer. In some embodiments, the mass spectrometer is a SCIEX X500R mass spectrometer. In some embodiments, the mass spectrometer is a SCIEX ZenoTOF 7600 mass spectrometer. In some embodiments, the mass spectrometer is a Shimadzu LCMS-9030 mass spectrometer. In some embodiments, the mass spectrometer is a Waters SELECT SERIES mass spectrometer. In some embodiments, the mass spectrometer is a Waters SYNAPT XS mass spectrometer. In some embodiments, the mass spectrometer is a Waters SELECT SERIES Cyclic IMS mass spectrometer. In some embodiments, the mass spectrometer is a Waters Xevo Gs-XS mass spectrometer. In some embodiments, the mass spectrometer is a Waters Vion IMS mass spectrometer.

The performance of a mass spectrometer is measured by its sensitivity, mass resolution and mass accuracy. Sensitivity is measured by the amount of material needed; it is generally desirable and possible with mass spectrometry to work with sample amounts in the femtomole and low picomole range. Mass resolution, m/Δm, is the measure of an instrument's ability to produce separate signals from ions of similar mass. Mass resolution is defined as the mass, m, of an ion signal divided by the full width of the signal, Δm, usually measured between points of half-maximum intensity. Mass accuracy is the measure of error in designating a mass to an ion signal. The mass accuracy is defined as the ratio of the mass assignment error divided by the mass of the ion and can be represented as a percentage. In some embodiments, the mass assignment error of the method is 0.01% or less, 0.009% or less, 0.008% or less, 0.007% or less, 0.006% or less, or 0.005% or less. In some embodiments, the method estimates the mass of a nucleic acid having a length between 300-500 nucleotides, 500-1000 nucleotides, 1000-1500 nucleotides, 1500-2000 nucleotides, 2000-2500 nucleotides, or 2500-3000 nucleotides, with a mass assignment error of 0.01% or less, 0.009% or less, 0.008% or less, 0.007% or less, 0.006% or less, or 0.005% or less. In some embodiments, the method estimates the mass of a nucleic acid having a length greater than 5000 nucleotides, with a mass assignment error of 0.01% or less, 0.009% or less, 0.008% or less, 0.007% or less, 0.006% or less, or 0.005% or less.

### Nucleic Acids

Aspects of the present disclosure relate to compositions comprising nucleic acids and methods of producing nucleic acids. As used herein, the term “nucleic acid” includes multiple nucleotides (i.e., molecules comprising a sugar (e.g., ribose or deoxyribose) linked to a phosphate group and to an exchangeable organic base, which is either a substituted pyrimidine (e.g., cytosine (C), thymine (T) or uracil (U)) or a substituted purine (e.g., adenine (A) or guanine (G))). The term nucleic acid includes polyribonucleotides as well as polydeoxyribonucleotides. The term nucleic acid also includes polynucleosides (i.e., a polynucleotide minus the phosphate) and any other organic base containing polymer. Non-limiting examples of nucleic acids include chromosomes, genomic loci, genes or gene segments that encode polynucleotides or polypeptides, coding sequences, non-coding sequences (e.g., intron, 5′-UTR, or 3′-UTR) of a gene, pri-mRNA, pre-mRNA, cDNA, mRNA, etc. A nucleic acid (e.g., mRNA) may include a substitution and/or modification. In some embodiments, the substitution and/or modification is in one or more bases and/or sugars. For example, in some embodiments a nucleic acid (e.g., mRNA) includes nucleotides having an organic group, such as a methyl group, attached to a nucleic acid base at the N6 position. Thus, in some embodiments, an mRNA includes one or more N6-methyladenosine nucleotides. A phosphate, sugar, or nucleic acid base of a nucleotide may also be substituted for another phosphate, sugar, or nucleic acid base. For example, a uridine base may be substituted for a pseudouridine base, in which the uracil base is attached to the sugar by a carbon-carbon bond rather than a nitrogen-carbon bond. Thus, in some embodiments, a nucleic acid (e.g., mRNA) is heterogeneous in backbone composition thereby containing any possible combination of polymer units linked together such as peptide-nucleic acids (which have an amino acid backbone with nucleic acid bases).

The nucleic acid sequences of the present invention include nucleic acid sequences that have been removed from their naturally occurring environment, recombinant or cloned DNA isolates, and chemically synthesized analogues or analogues biologically synthesized by heterologous systems.

An “engineered nucleic acid” is a nucleic acid that does not occur in nature. It should be understood, however, that while an engineered nucleic acid as a whole is not naturally-occurring, it may include nucleotide sequences that occur in nature. In some embodiments, an engineered nucleic acid comprises nucleotide sequences from different organisms (e.g., from different species). For example, in some embodiments, an engineered nucleic acid includes a bacterial nucleotide sequence, a human nucleotide sequence, and/or a viral nucleotide sequence.

Engineered nucleic acids include recombinant nucleic acids and synthetic nucleic acids. A “recombinant nucleic acid” is a molecule that is constructed by joining nucleic acids (e.g., isolated nucleic acids, synthetic nucleic acids or a combination thereof) and, in some embodiments, can replicate in a living cell. A “synthetic nucleic acid” is a molecule that is amplified or chemically, or by other means, synthesized. A synthetic nucleic acid includes those that are chemically modified, or otherwise modified, but can base pair with naturally-occurring nucleic acid molecules. Recombinant and synthetic nucleic acids also include those molecules that result from the replication of either of the foregoing. A nucleic may comprise naturally occurring nucleotides and/or non-naturally occurring nucleotides such as modified nucleotides.

In some embodiments, a nucleic acid is present in (or on) a vector. Examples of vectors include but are not limited to bacterial plasmids, phage, cosmids, phasmids, fosmids, bacterial artificial chromosomes, yeast artificial chromosomes, viruses and retroviruses (for example vaccinia, adenovirus, adeno-associated virus, lentivirus, herpes-simplex virus, Epstein-Barr virus, fowlpox virus, pseudorabies, baculovirus) and vectors derived therefrom. In some embodiments, a nucleic acid (e.g., DNA) used as an input molecule for in vitro transcription (IVT) is present in a plasmid vector.

When applied to a nucleic acid sequence, the term “isolated” denotes that the polynucleotide sequence has been removed from its natural genetic milieu and is thus free of other extraneous or unwanted coding sequences (but may include naturally occurring 5′ and 3′ untranslated regions such as promoters and terminators), and is in a form suitable for use within genetically engineered protein production systems. Such isolated molecules are those that are separated from their natural environment.

In some embodiments, an input DNA for IVT is a nucleic acid vector. A “nucleic acid vector” is a polynucleotide that carries at least one foreign or heterologous nucleic acid fragment. A nucleic acid vector may function like a “molecular carrier”, delivering fragments of nucleic acids or polynucleotides into a host cell or as a template for IVT. An “in vitro transcription template” (IVT template), or “input DNA” as used herein, refers to deoxyribonucleic acid (DNA) suitable for use in an IVT reaction for the production of messenger RNA (mRNA). In some embodiments, an IVT template encodes a 5′ untranslated region, contains an open reading frame, and encodes a 3′ untranslated region and a polyA tail. The particular nucleotide sequence composition and length of an IVT template will depend on the mRNA of interest encoded by the template.

In some embodiments the nucleic acid vector according to the invention is a circular nucleic acid such as a plasmid. In other embodiments it is a linearized nucleic acid. According to some embodiments the nucleic acid vector comprises a predefined restriction site, which can be used for linearization. The linearization restriction site determines where the vector nucleic acid is opened/linearized. The restriction enzymes chosen for linearization should preferably not cut within the critical components of the vector.

A nucleic acid vector may include an insert which may be an expression cassette or open reading frame (ORF). An “open reading frame” is a continuous stretch of DNA beginning with a start codon (e.g., methionine (ATG)), and ending with a stop codon (e.g., TAA, TAG or TGA) and encodes a protein or peptide (e.g., a therapeutic protein or therapeutic peptide). In some embodiments, an expression cassette encodes an RNA including at least the following elements: a 5′ untranslated region, an open reading frame region encoding the mRNA, a 3′ untranslated region and a polyA tail. The open reading frame may encode any mRNA sequence, or portion thereof.

In some embodiments, a nucleic acid vector comprises a 5′ untranslated region (UTR). A “5′ untranslated region (UTR)” refers to a region of an mRNA that is directly upstream (i.e., 5′) from the start codon (i.e., the first codon of an mRNA transcript translated by a ribosome) that does not encode a protein or peptide. 5′ UTRs are further described herein, for example in the section entitled “Untranslated Regions”.

In some embodiments, a nucleic acid vector comprises a 3′ untranslated region (UTR). A “3′ untranslated region (UTR)” refers to a region of an mRNA that is directly downstream (i.e., 3′) from the stop codon (i.e., the codon of an mRNA transcript that signals a termination of translation) that does not encode a protein or peptide. 3′ UTRs are further described herein, for example in the section entitled “Untranslated Regions”.

The terms 5′ and 3′ are used herein to describe features of a nucleic acid sequence related to either the position of genetic elements and/or the direction of events (5′ to 3′), such as e.g. transcription by RNA polymerase or translation by the ribosome which proceeds in 5′ to 3′ direction. Synonyms are upstream (5′) and downstream (3′). Conventionally, DNA sequences, gene maps, vector cards and RNA sequences are drawn with 5′ to 3′ from left to right or the 5′ to 3′ direction is indicated with arrows, wherein the arrowhead points in the 3′ direction. Accordingly, 5′ (upstream) indicates genetic elements positioned towards the left-hand side, and 3′ (downstream) indicates genetic elements positioned towards the right-hand side, when following this convention.

Aspects of the disclosure relate to populations of molecules. As used herein, a “population” of molecules (e.g., DNA molecules) generally refers to a preparation (e.g., a plasmid preparation) comprising a plurality of copies of the molecule (e.g., DNA) of interest, for example a cell extract preparation comprising a plurality of expression vectors encoding a molecule of interest (e.g., a DNA encoding an RNA of interest).

A nucleic acid (e.g., mRNA) typically comprises a plurality of nucleotides. A nucleotide includes a nitrogenous base, a five-carbon sugar (ribose or deoxyribose), and at least one phosphate group. Nucleotides include nucleoside monophosphates, nucleoside diphosphates, and nucleoside triphosphates. A nucleoside monophosphate (NMP) includes a nucleobase linked to a ribose and a single phosphate; a nucleoside diphosphate (NDP) includes a nucleobase linked to a ribose and two phosphates; and a nucleoside triphosphate (NTP) includes a nucleobase linked to a ribose and three phosphates. Nucleotide analogs are compounds that have the general structure of a nucleotide or are structurally similar to a nucleotide. Nucleotide analogs, for example, include an analog of the nucleobase, an analog of the sugar and/or an analog of the phosphate group(s) of a nucleotide.

A nucleoside includes a nitrogenous base and a 5-carbon sugar. Thus, a nucleoside plus a phosphate group yields a nucleotide. Nucleoside analogs are compounds that have the general structure of a nucleoside or are structurally similar to a nucleoside. Nucleoside analogs, for example, include an analog of the nucleobase and/or an analog of the sugar of a nucleoside.

It should be understood that the term “nucleotide” includes naturally-occurring nucleotides, synthetic nucleotides and modified nucleotides, unless indicated otherwise. Examples of naturally-occurring nucleotides used for the production of RNA, e.g., in an IVT reaction, as provided herein include adenosine triphosphate (ATP), guanosine triphosphate (GTP), cytidine triphosphate (CTP), uridine triphosphate (UTP), and 5-methyluridine triphosphate (m⁵UTP). In some embodiments, adenosine diphosphate (ADP), guanosine diphosphate (GDP), cytidine diphosphate (CDP), and/or uridine diphosphate (UDP) are used.

Examples of nucleotide analogs include, but are not limited to, antiviral nucleotide analogs, phosphate analogs (soluble or immobilized, hydrolyzable or non-hydrolyzable), dinucleotide, trinucleotide, tetranucleotide, e.g., a cap analog, or a precursor/substrate for enzymatic capping (vaccinia or ligase), a nucleotide labeled with a functional group to facilitate ligation/conjugation of cap or 5′ moiety (IRES), a nucleotide labeled with a 5′ PO₄ to facilitate ligation of cap or 5′ moiety, or a nucleotide labeled with a functional group/protecting group that can be chemically or enzymatically cleaved. Examples of antiviral nucleotide/nucleoside analogs include, but are not limited, to Ganciclovir, Entecavir, Telbivudine, Vidarabine and Cidofovir.

Modified nucleotides may include modified nucleobases or sugars. For example, an RNA transcript (e.g., mRNA transcript) of the present disclosure may include a modified nucleoside selected from pseudouridine (W), 1-methylpseudouridine (m1ψ), 1-ethylpseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine (mo5U) and 2′-O-methyluridine. In some embodiments, an RNA transcript (e.g., mRNA transcript) includes a combination of at least two (e.g., 2, 3, 4 or more) of the foregoing modified nucleosides.

In some embodiments, a target RNA analyzed by a method described herein is a circular mRNA. A circular RNA is an RNA with no 5′ terminal nucleotide or 3′ terminal nucleotide. Every nucleotide in a circular RNA is covalently bonded to both (i) a 5′ adjacent nucleotide; and (ii) a 3′ adjacent nucleotide. In a circular RNA with a nucleic acid sequence comprising every nucleotide of the circular RNA in 5′-to-3′ order, the last nucleotide of the nucleic acid sequence is covalently bonded to the first nucleotide of the nucleic acid sequence. Circular mRNAs may be produced by multiple methods known in the art, such as by forming a covalent bond between two non-adjacent nucleotides of a linear mRNA. For example, a circular mRNA may be formed by ligating a 5′ terminal nucleotide and a 3′ terminal nucleotide of the linear mRNA using an RNA ligase. The RNA ligase may be any RNA ligase known in the art, such as T4 RNA ligase, SplintR ligase, or RtcB ligase. For ligation to occur, the 5′ and 3′ terminal nucleotides of the mRNA must be close enough for the RNA ligase to form a bond between both nucleotides. Methods of placing both nucleotides of a linear nucleic acid close enough for ligation to occur, and of circularizing an RNA, are generally known in the art. See, e.g., Petkovic et al., Nucleic Acids Res. 2015. 43(4):2454-2465. Non-limiting examples of circularization methods include splinted ligation and ribozyme-mediated circularization. In splinted ligation, a nucleic acid to be ligated (e.g., linear mRNA), is contacted with a splint nucleic acid, such as a DNA oligonucleotide, which hybridizes to 5′ and 3′ terminal sequences, such that hybridization places the 5′ and 3′ terminal nucleotide in close proximity, allowing for ligation by an RNA ligase. After forming this structure, the RNA:splint nucleic acid is contacted with an RNA ligase that forms a covalent bond between the 5′ terminal nucleotide and the 3′ terminal nucleotide of the RNA. A ribozyme is a nucleic acid that catalyzes a reaction, such as the formation of a covalent bond between two nucleotides. For example, prior to circularization, an mRNA may comprise a 3′ intron that is 5′ to (upstream of) the 5′ UTR of the mRNA, and a 5′ intron that is 3′ to (downstream of) the poly-A region and/or one or more structural sequences of the mRNA. Ribozymes and other enzymes that catalyze splicing of pre-mRNA to remove introns can catalyze the formation of a covalent bond between the nucleotide that is upstream from the 5′ intron and the nucleotide that is downstream from 3′ intron, resulting in the formation of a circular mRNA. See, e.g., Wesselhoeft et al., Nat Commun. 2018. 9:2629.

In some embodiments, the circular mRNA comprises an internal ribosome entry site (IRES). Because circular mRNAs have no 5′ terminal end, and thus no 5′ cap, to allow cap-mediated ribosome recruitment, translation initiation may be achieved by alternative means. An IRES is a nucleotide sequence located within a nucleic acid (i.e., not at a 5′ or 3′ terminal end), to which a ribosome can bind, and thereafter initiate translation. See, e.g., Yakubov et al., Biochem Biophys Res Commun. 2010. 394(1):189-193. In some embodiments, a circular mRNA 5 comprises, in 5′-to-3′ order, a 5′ untranslated region (UTR), an open reading frame encoding a polypeptide, and a 3′ UTR. In some embodiments, the circular mRNA further comprises a polyA region. In some embodiments, the polyA region is downstream from the 3′ UTR. In some embodiments, one or more intervening sequences (e.g., a second open reading frame) are located between the 3′ UTR and the polyA region. In some embodiments, the polyA region is upstream from the 5′ UTR. In some embodiments, one or more intervening sequences (e.g., an IRES) are located between the polyA region and the 5′ UTR. In some embodiments, the circular mRNA comprises, in 5′-to-3′ order: the 3′ UTR, the polyA region, and the 5′ UTR.

In vitro Transcription

Aspects of the present disclosure provide methods of producing (e.g., synthesizing) an RNA transcript (e.g., mRNA transcript) comprising contacting a DNA template (e.g., a first input DNA and a second input DNA) with an RNA polymerase (e.g., a T7 RNA polymerase, a T7 RNA polymerase variant, etc.) under conditions that result in the production of the RNA transcript. This process is referred to as “in vitro transcription” or “IVT”. IVT conditions typically require a purified DNA template containing a promoter, nucleoside triphosphates, a buffer system that includes dithiothreitol (DTT) and magnesium ions, and an RNA polymerase. The exact conditions used in the transcription reaction depend on the amount of RNA needed for a specific application. Typical IVT reactions are performed by incubating a DNA template with an RNA polymerase and nucleoside triphosphates, including GTP, ATP, CTP, and UTP (or nucleotide analogs) in a transcription buffer. An RNA transcript having a 5′ terminal guanosine triphosphate is produced from this reaction.

In some embodiments, an IVT reaction uses an RNA polymerase selected from the group consisting of T7 RNA polymerase, T3 RNA polymerase, K11 RNA polymerase, and SP6 RNA polymerase. In some embodiments, an IVT reaction uses a T3 RNA polymerase. In some embodiments, an IVT reaction uses an SP6 RNA polymerase. In some embodiments, an IVT reaction uses a K11 RNA polymerase. In some embodiments, an IVT reaction uses a T7 RNA polymerase. In some embodiments, a wild-type T7 polymerase is used in an IVT reaction. In some embodiments, a mutant T7 polymerase is used in an IVT reaction. In some embodiments, a T7 RNA polymerase variant comprises an amino acid sequence that shares at least 50%, 60%, 70%, 80%, 90%, 95%, or 99% identity with a wild-type T7 (WT T7) polymerase. In some embodiments, the T7 polymerase variant is a T7 polymerase variant described by International Application Publication Number WO2019/036682 or WO2020/172239, the entire contents of each of which are incorporated herein by reference. T7 RNA polymerase variants with one or more mutations relative to WT T7 RNA polymerase have several advantages in IVT reactions, including improved speed, fidelity, and reduced production of double-stranded RNA (dsRNA) transcripts. Double-stranded RNA transcripts, in which at least a portion of an RNA transcript is hybridized to another RNA molecule, elicit an innate immune response when introduced into a cell, causing degradation of both strands of a dsRNA. Minimizing the formation of dsRNA transcripts during IVT enables the production of less immunogenic, and thus more stable, RNA compositions. In some embodiments, the concentration of double-stranded RNA in a composition comprising RNA is 5% (% w/w) or less, 4% or less, 3% or less, 2.5% or less, 2% or less, 1.75% or less, 1.5% or less, 1.25% or less, 1% or less, 0.9% or less, 0.8% or less, 0.7% or less, 0.6% or less, 0.5% or less, 0.4% or less, 0.3% or less, 0.25% or less, 0.2% or less, 0.175% or less, 0.15% or less, 0.125% or less, or 0.1% or less. In some embodiments, the concentration of double-stranded RNA in a composition comprising RNA is 0.05% (% w/w) or less, 0.04% or less, 0.03% or less, 0.02% or less, or 0.01% or less. Methods of measuring the presence and/or amount of dsRNA in a composition are known in the art. Non-limiting examples of methods for measuring dsRNA content of a sample include ELISAs and immunoblotting using antibodies specific to dsRNA. Additionally, the total mass of RNA in a sample can be measured using techniques such as spectroscopy (NanoDrop), qRT-PCR, and/or ddPCR, and the mass of dsRNA can be measured using an intercalating agent that fluoresces when bound to dsRNA, such as acridine orange, with the dsRNA concentration being calculated by division. In some embodiments, the concentration of dsRNA in a composition refers to the mass of RNA nucleotides that are part of a double-stranded RNA:RNA hybrid, with other unhybridized nucleotides from either RNA in the hybrid not contributing to the amount of dsRNA in a composition. In other embodiments, the concentration of dsRNA in a sample refers to the concentration of RNA molecules containing nucleotides that are part of an RNA:RNA hybrid. In some embodiments, the RNA polymerase (e.g., T7 RNA polymerase or T7 RNA polymerase variant) is present in a reaction (e.g., an IVT reaction) at a concentration of 0.01 mg/ml to 1 mg/ml. For example, the RNA polymerase may be present in a reaction at a concentration of 0.01 mg/mL, 0.05 mg/ml, 0.1 mg/ml, 0.5 mg/ml or 1.0 mg/ml.

The “percent identity,” “sequence identity,” “% identity,” or “% sequence identity” (as they may be interchangeably used herein) of two sequences (e.g., nucleic acid or amino acid) refers to a quantitative measurement of the similarity between two sequences (e.g., nucleic acid or amino acid). Percent identity can be determined using the algorithms of Karlin and Altschul, Proc. Natl. Acad. Sci. USA 87:2264-68, 1990, modified as in Karlin and Altschul, Proc. Natl. Acad. Sci. USA 90:5873-77, 1993. Such algorithms are incorporated into the NBLAST and XBLAST programs (version 2.0) of Altschul et al., J. Mol. Biol. 215:403-10, 1990. BLAST protein searches can be performed with the XBLAST program, score=50, word length=3, to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used. When a percent identity is stated, or a range thereof (e.g., at least, more than, etc.), unless otherwise specified, the endpoints shall be inclusive and the range (e.g., at least 70% identity) shall include all ranges within the cited range.

The input deoxyribonucleic acid (DNA) serves as a nucleic acid template for RNA polymerase. A DNA template may include a polynucleotide encoding a polypeptide of interest (e.g., an antigenic polypeptide). A DNA template, in some embodiments, includes an RNA polymerase promoter (e.g., a T7 RNA polymerase promoter) located 5′ from and operably linked to polynucleotide encoding a polypeptide of interest. A DNA template may also include a nucleotide sequence encoding a polyadenylation (polyA) region located at the 3′ end of the gene of interest. In some embodiments, an input DNA comprises plasmid DNA (pDNA). As used herein, “plasmid DNA” or “pDNA” refers to an extrachromosomal DNA molecule that is physically separated from chromosomal DNA in a cell and can replicate independently. In some embodiments, plasmid DNA is isolated from a cell (e.g., as a plasmid DNA preparation). In some embodiments, plasmid DNA comprises an origin of replication, which may contain one or more heterologous nucleic acids, for example nucleic acids encoding therapeutic proteins that may serve as a template for RNA polymerase. Plasmid DNA may be circularized or linear (e.g., plasmid DNA that has been linearized by a restriction enzyme digest).

Some embodiments comprise performing a co-IVT reaction that includes multiple input DNAs (or populations of input DNAs). In some embodiments, each input DNA (e.g., population of input DNA molecules) in a co-IVT reaction is obtained from a different source (e.g., synthesized separately, for example in different cells or populations of cells). In some embodiments, each input DNA (e.g., population of input DNA) is obtained from a different bacterial cell or population of bacterial cells. For example, in a co-IVT reaction having three populations of input DNAs, the first input DNA is produced in bacterial cell population A, the second input DNA is produced in bacterial cell population B, and the third input DNA is produced in bacterial population C, where each of A, B, and C are not the same bacterial culture (e.g., co-cultured in the same container or plate). In another example, two input DNAs obtained from different sources are i) chemically synthesized in separate synthesis reactions, or ii) produced by separate amplification (e.g., polymerase chain reactions (PCR reactions)).

An RNA transcript, in some embodiments, is the product of an IVT reaction. An RNA transcript, in some embodiments, is a messenger RNA (mRNA) that includes a nucleotide sequence encoding a polypeptide of interest (e.g., a therapeutic protein or therapeutic peptide) linked to a polyA tail. In some embodiments, the mRNA is modified mRNA (mmRNA), which includes at least one modified nucleotide. In some embodiments, an RNA transcript produced by IVT is further modified by circularization, in which two non-adjacent nucleotides (e.g., 5′ and 3′ terminal nucleotides) of a linear RNA are ligated to produce a circular RNA with no terminal nucleotides.

The nucleoside triphosphates (NTPs) as provided herein may comprise unmodified or modified ATP, modified or unmodified UTP, modified or unmodified GTP, and/or modified or unmodified CTP. In some embodiments, NTPs of an IVT reaction comprise unmodified ATP. In some embodiments, NTPs of an IVT reaction comprise modified ATP. In some embodiments, NTPs of an IVT reaction comprise unmodified UTP. In some embodiments, NTPs of an IVT reaction comprise modified UTP. In some embodiments, NTPs of an IVT reaction comprise unmodified GTP. In some embodiments, NTPs of an IVT reaction comprise modified GTP. In some embodiments, NTPs of an IVT reaction comprise unmodified CTP. In some embodiments, NTPs of an IVT reaction comprise modified CTP.

The composition of NTPs in an IVT reaction may also vary. In some embodiments, each NTP in an IVT reaction is present in an equimolar amount. In some embodiments, each NTP in an IVT reaction is present in non-equimolar amounts. For example, ATP may be used in excess of GTP, CTP and UTP. As a non-limiting example, an IVT reaction may include 7.5 millimolar GTP, 7.5 millimolar CTP, 7.5 millimolar UTP, and 3.75 millimolar ATP. In some embodiments, the molar ratio of G:C:U:A is 2:1:0.5:1. In some embodiments, the molar ratio of G:C:U:A is 1:1:0.7:1. In some embodiments, the molar ratio of G:C:A:U is 1:1:1:1. The same IVT reaction may include 3.75 millimolar cap analog (e.g., trinucleotide cap or tetranucleotide cap). In some embodiments, the molar ratio of the cap to any of G, C, U, or A is 1:1. In some embodiments, the molar ratio of G:C:U:A:cap is 1:1:1:0.5:0.5. In some embodiments, the molar ratio of G:C:U:A:cap is 1:1:0.5:1:0.5. In some embodiments, the molar ratio of G:C:U:A:cap is 1:0.5:1:1:0.5. In some embodiments, the molar ratio of G:C:U:A:cap is 0.5:1:1:1:0.5. In some embodiments, the amount of NTPs in a IVT reaction is calculated empirically. For example, the rate of consumption for each NTP in an IVT reaction may be empirically determined for each individual input DNA, and then balanced ratios of NTPs based on those individual NTP consumption rates may be added to a IVT comprising multiple of the input DNAs.

In some embodiments, the IVT reaction mixture comprises one or more modified nucleoside triphosphates. In some embodiments, the IVT reaction mixture comprises one or more modified nucleoside triphosphates selected from the group consisting of N6-methyladenosine triphosphate, pseudouridine (ψ) triphosphate, 1-methylpseudouridine (m1ψ) triphosphate, 5-methoxyuridine (mo⁵U) triphosphate, 5-methylcytidine (m⁵C) triphosphate, α-thio-guanosine triphosphate, and α-thio-adenosine triphosphate. In some embodiments, the IVT reaction mixture comprises N6-methyladenosine triphosphate. In some embodiments, the IVT reaction mixture comprises pseudouridine triphosphate. In some embodiments, the IVT reaction mixture comprises 1-methylpseudouridine triphosphate. In some embodiments, the concentration of modified nucleoside triphosphates in the reaction mixture is about 0.1% to about 100%, about 0.5% to about 75%, about 1% to about 50%, or about 2% to about 25%. In some embodiments, the concentration of modified nucleoside triphosphates is about 1%, about 2%, about 3%, about 4%, about 5%, about 6%, about 7%, about 8%, about 9%, about 10%, about 15%, about 20%, or about 25%.

In some embodiments, an RNA transcript (e.g., mRNA transcript) includes a modified nucleoside selected from pseudouridine (W), 1-methylpseudouridine (m¹ψ), 5-methoxyuridine (mo⁵U), 5-methylcytidine (m⁵C), α-thio-guanosine and α-thio-adenosine. In some embodiments, an RNA transcript (e.g., mRNA transcript) includes a combination of at least two (e.g., 2, 3, 4 or more) of the foregoing modified nucleosides.

In some embodiments, an RNA transcript (e.g., mRNA transcript) includes pseudouridine (ψ). In some embodiments, an RNA transcript (e.g., mRNA transcript) includes 1-methylpseudouridine (m1ψ). In some embodiments, an RNA transcript (e.g., mRNA transcript) includes 5-methoxyuridine (mo⁵U). In some embodiments, an RNA transcript (e.g., mRNA transcript) includes 5-methylcytidine (m⁵C). In some embodiments, an RNA transcript (e.g., mRNA transcript) includes α-thio-guanosine. In some embodiments, an RNA transcript (e.g., mRNA transcript) includes α-thio-adenosine.

In some embodiments, the polynucleotide (e.g., RNA polynucleotide, such as mRNA polynucleotide) is uniformly modified (e.g., fully modified, modified throughout the entire sequence) for a particular modification. For example, a polynucleotide can be uniformly modified with 1-methylpseudouridine (m¹ψ), meaning that all uridine residues in the mRNA sequence are replaced with 1-methylpseudouridine (m¹ψ). Similarly, a polynucleotide can be uniformly modified for any type of nucleoside residue present in the sequence by replacement with a modified residue such as any of those set forth above. Alternatively, the polynucleotide (e.g., RNA polynucleotide, such as mRNA polynucleotide) may not be uniformly modified (e.g., partially modified, part of the sequence is modified). Each possibility represents a separate embodiment of the present invention. In some embodiments, modified nucleotides are included in an IVT mixture, and are incorporated randomly during transcription, such that the RNA contains a mixture of modified nucleotides and unmodified nucleotides.

The buffer system of an IVT reaction mixture may vary. In some embodiments, the buffer system contains Tris. The concentration of tris used in an IVT reaction, for example, may be at least 10 mM, at least 20 mM, at least 30 mM, at least 40 mM, at least 50 mM, at least 60 mM, at least 70 mM, at least 80 mM, at least 90 mM, at least 100 mM or at least 110 mM phosphate. In some embodiments, the concentration of phosphate is 20-60 mM or 10-100 mM. In some embodiments, the buffer system contains dithiothreitol (DTT). The concentration of DTT used in an IVT reaction, for example, may be at least 1 mM, at least 5 mM, or at least 50 mM. In some embodiments, the concentration of DTT used in an IVT reaction is 1-50 mM or 5-50 mM. In some embodiments, the concentration of DTT used in an IVT reaction is 5 mM. In some embodiments, the buffer system contains magnesium. In some embodiments, the molar ratio of NTP to magnesium ions (Mg²⁺; e.g., MgCl₂) present in an IVT reaction is 1:1 to 1:5. For example, the molar ratio of NTP to magnesium ions may be 1:0.25, 1:0.5, 1:1, 1:2, 1:3, 1:4 or 1:5.

In some embodiments, the molar ratio of NTP to magnesium ions (Mg²⁺; e.g., MgCl₂) present in an IVT reaction is 1:1 to 1:5. For example, the molar ratio of NTP to magnesium ions may be 1:1, 1:2, 1:3, 1:4 or 1:5.

In some embodiments, the buffer system contains Tris-HCl, spermidine (e.g., at a concentration of 1-30 mM), TRITON® X-100 (polyethylene glycol p-(1,1,3,3-tetramethylbutyl)-phenyl ether) and/or polyethylene glycol (PEG).

In some embodiments, IVT methods further comprise a step of separating (e.g., purifying) in vitro transcription products (e.g., mRNA) from other reaction components. In some embodiments, the separating comprises performing chromatography on the IVT reaction mixture. In some embodiments, the method comprises reverse phase chromatography. In some embodiments, the method comprises reverse phase column chromatography. In some embodiments, the chromatography comprises size-based (e.g., length-based) chromatography. In some embodiments, the method comprises size exclusion chromatography. In some embodiments, the chromatography comprises oligo-dT chromatography.

### Untranslated Regions

Untranslated regions (UTRs) are sections of a nucleic acid before a start codon (5′ UTR) and after a stop codon (3′ UTR) that are not translated. In some embodiments, a nucleic acid (e.g., a ribonucleic acid (RNA), e.g., a messenger RNA (mRNA)) of the disclosure comprising an open reading frame (ORF) encoding one or more proteins or peptides further comprises one or more UTR (e.g., a 5′ UTR or functional fragment thereof, a 3′ UTR or functional fragment thereof, or a combination thereof).

A UTR can be homologous or heterologous to the coding region in a nucleic acid. In some embodiments, the UTR is homologous to the ORF encoding the one or more peptide epitopes. In some embodiments, the UTR is heterologous to the ORF encoding the one or more peptide epitopes. In some embodiments, the nucleic acid comprises two or more 5′ UTRs or functional fragments thereof, each of which have the same or different nucleotide sequences. In some embodiments, the nucleic acid comprises two or more 3′ UTRs or functional fragments thereof, each of which have the same or different nucleotide sequences.

In some embodiments, the 5′ UTR or functional fragment thereof, 3′ UTR or functional fragment thereof, or any combination thereof is sequence optimized.

In some embodiments, the 5′ UTR or functional fragment thereof, 3′ UTR or functional fragment thereof, or any combination thereof comprises at least one chemically modified nucleobase, e.g., 5-methoxyuracil.

UTRs can have features that provide a regulatory role, e.g., increased or decreased stability, localization, and/or translation efficiency. A nucleic acid comprising a UTR can be administered to a cell, tissue, or organism, and one or more regulatory features can be measured using routine methods. In some embodiments, a functional fragment of a 5′ UTR or 3′ UTR comprises one or more regulatory features of a full length 5′ or 3′ UTR, respectively.

Natural 5′ UTRs bear features that play roles in translation initiation. They harbor signatures like Kozak sequences that are commonly known to be involved in the process by which the ribosome initiates translation of many genes. 5′ UTRs also have been known to form secondary structures that are involved in elongation factor binding. mRNAs may also comprise a 5′ cap upstream from the 5′ UTR. In some embodiments, an mRNA comprises a 7-methylguanosine cap or a 7-methylguanosine group analog (e.g., a cap analog as described by Kowalska et al., RNA. 2008. 14(6):1119-1131).

By engineering the features typically found in abundantly expressed genes of specific target organs, one can enhance the stability and protein production of a nucleic acid. For example, introduction of 5′ UTR of liver-expressed mRNA, such as albumin, serum amyloid A, Apolipoprotein A/B/E, transferrin, alpha fetoprotein, erythropoietin, or Factor VIII, can enhance expression of nucleic acids in hepatic cell lines or liver. Likewise, use of 5′ UTRs from other tissue-specific mRNA to improve expression in that tissue is possible for muscle (e.g., MyoD, Myosin, Myoglobin, Myogenin, Herculin), for endothelial cells (e.g., Tie-1, CD36), for myeloid cells (e.g., C/EBP, AML1, G-CSF, GM-CSF, CD11b, MSR, Fr-1, i-NOS), for leukocytes (e.g., CD45, CD18), for adipose tissue (e.g., CD36, GLUT4, ACRP30, adiponectin), and for lung epithelial cells (e.g., SP-A/B/C/D).

In some embodiments, UTRs are selected from a family of transcripts whose proteins share a common function, structure, feature, or property. For example, an encoded polypeptide can belong to a family of proteins (i.e., that share at least one function, structure, feature, localization, origin, or expression pattern), which are expressed in a particular cell, tissue or at some time during development. The UTRs from any of the genes or mRNA can be swapped for any other UTR of the same or different family of proteins to create a new nucleic acid. In some embodiments, the 5′ UTR and the 3′ UTR can be heterologous. In some embodiments, the 5′ 10 UTR can be derived from a different species than the 3′ UTR. In some embodiments, the 3′ UTR can be derived from a different species than the 5′ UTR.

International Patent Application No. PCT/US2014/021522 (Publ. No. WO/2014/164253) provides a listing of exemplary UTRs that may be utilized in the nucleic acids of the present disclosure as flanking regions to an ORF. This publication is incorporated by reference herein for this purpose.

Additional exemplary UTRs that may be utilized in the nucleic acids of the present disclosure include, but are not limited to, one or more 5′ UTRs and/or 3′ UTRs derived from the nucleic acid sequence of: a globin, such as an α- or β-globin (e.g., a Xenopus, mouse, rabbit, or human globin); a strong Kozak translational initiation signal; a CYBA (e.g., human cytochrome b-245 α polypeptide); an albumin (e.g., human albumin7); a HSD17B4 (hydroxysteroid (17-β) dehydrogenase); a virus (e.g., a tobacco etch virus (TEV), a Venezuelan equine encephalitis virus (VEEV), a Dengue virus, a cytomegalovirus (CMV; e.g., CMV immediate early 1 (IE1)), a hepatitis virus (e.g., hepatitis B virus), a sindbis virus, or a PAV barley yellow dwarf virus); a heat shock protein (e.g., hsp70); a translation initiation factor (e.g., eIF4G); a glucose transporter (e.g., hGLUT1 (human glucose transporter 1)); an actin (e.g., human α or β actin); a GAPDH; a tubulin; a histone; a citric acid cycle enzyme; a topoisomerase (e.g., a 5′ UTR of a TOP gene lacking the 5′ TOP motif (the oligopyrimidine tract)); a ribosomal protein Large 32 (L32); a ribosomal protein (e.g., human or mouse ribosomal protein, such as, for example, rps9); an ATP synthase (e.g., ATP5A1 or the β subunit of mitochondrial H⁺-ATP synthase); a growth hormone (e.g., bovine (bGH) or human (hGH)); an elongation factor (e.g., elongation factor 1 α1 (EEF1A1)); a manganese superoxide dismutase (MnSOD); a myocyte enhancer factor 2A (MEF2A); a β-F1-ATPase, a creatine kinase, a myoglobin, a granulocyte-colony stimulating factor (G-CSF); a collagen (e.g., collagen type I, alpha 2 (CollA2), collagen type I, alpha 1 (CollA1), collagen type VI, alpha 2 (Col6A2), collagen type VI, alpha 1 (Col6A1)); a ribophorin (e.g., ribophorin I (RPNI)); a low density lipoprotein receptor-related protein (e.g., LRP1); a cardiotrophin-like cytokine factor (e.g., Nnt1); calreticulin (Calr); a procollagen-lysine, 2-oxoglutarate 5-dioxygenase 1 (Plod1); and a nucleobindin (e.g., Nucb1).

In some embodiments, the 5′ UTR is selected from the group consisting of a β-globin 5′ UTR; a 5′ UTR containing a strong Kozak translational initiation signal; a cytochrome b-245 a polypeptide (CYBA) 5′ UTR; a hydroxysteroid (17-β) dehydrogenase (HSD17B4) 5′ UTR; a Tobacco etch virus (TEV) 5′ UTR; a Venezuelen equine encephalitis virus (TEEV) 5′ UTR; a 5′ proximal open reading frame of rubella virus (RV) RNA encoding nonstructural proteins; a Dengue virus (DEN) 5′ UTR; a heat shock protein 70 (Hsp70) 5′ UTR; a eIF4G 5′ UTR; a GLUT1 5′ UTR; functional fragments thereof and any combination thereof.

In some embodiments, the 3′ UTR is selected from the group consisting of a β-globin 3′ UTR; a CYBA 3′ UTR; an albumin 3′ UTR; a growth hormone (GH) 3′ UTR; a VEEV 3′ UTR; a hepatitis B virus (HBV) 3′ UTR; α-globin 3′ UTR; a DEN 3′ UTR; a PAV barley yellow dwarf virus (BYDV-PAV) 3′ UTR; an elongation factor 1 α1 (EEF1A1) 3′ UTR; a manganese superoxide dismutase (MnSOD) 3′ UTR; a β subunit of mitochondrial H(+)-ATP synthase ((β-mRNA) 3′ UTR; a GLUT1 3′ UTR; a MEF2A 3′ UTR; a β-F1-ATPase 3′ UTR; functional fragments thereof and combinations thereof.

Wild-type UTRs derived from any gene or mRNA can be incorporated into the nucleic acids of the disclosure. In some embodiments, a UTR can be altered relative to a wild type or native UTR to produce a variant UTR, e.g., by changing the orientation or location of the UTR relative to the ORF; or by inclusion of additional nucleotides, deletion of nucleotides, swapping or transposition of nucleotides. In some embodiments, variants of 5′ or 3′ UTRs can be utilized, for example, mutants of wild type UTRs, or variants wherein one or more nucleotides are added to or removed from a terminus of the UTR.

Additionally, one or more synthetic UTRs can be used in combination with one or more 25 non-synthetic UTRs. See, e.g., Mandal and Rossi, Nat. Protoc. 2013 8(3):568-82, and sequences available at www.addgene.org, the contents of each are incorporated herein by reference in their entirety. UTRs or portions thereof can be placed in the same orientation as in the transcript from which they were selected or can be altered in orientation or location. Hence, a 5′ and/or 3′ UTR can be inverted, shortened, lengthened, or combined with one or more other 5′ UTRs or 3′ UTRs.

In some embodiments, the nucleic acid may comprise multiple UTRs, e.g., a double, a triple or a quadruple 5′ UTR or 3′ UTR. For example, a double UTR comprises two copies of the same UTR either in series or substantially in series. For example, a double beta-globin 3′ UTR can be used (see, for example, US2010/0129877, the contents of which are incorporated herein by reference for this purpose).

The nucleic acids of the disclosure can comprise combinations of features. For example, the ORF can be flanked by a 5′ UTR that comprises a strong Kozak translational initiation signal and/or a 3′ UTR comprising an oligo(dT) sequence for templated addition of a polyA tail. A 5′ UTR can comprise a first nucleic acid fragment and a second nucleic acid fragment from the same and/or different UTRs (see, e.g., US2010/0293625, herein incorporated by reference in its entirety for this purpose).

Other non-UTR sequences can be used as regions or subregions within the nucleic acids of the disclosure. For example, introns or portions of intron sequences can be incorporated into the nucleic acids of the disclosure. Incorporation of intronic sequences can increase protein production as well as nucleic acid expression levels. In some embodiments, the nucleic acid of the disclosure comprises an internal ribosome entry site (IRES) instead of or in addition to a UTR (see, e.g., Yakubov et al., Biochem. Biophys. Res. Commun. 2010 394(1):189-193, the contents of which are incorporated herein by reference in their entirety). In some embodiments, the nucleic acid comprises an IRES instead of a 5′ UTR sequence. In some embodiments, the nucleic acid comprises an IRES that is located between a 5′ UTR and an open reading frame. In some embodiments, the nucleic acid comprises an ORF encoding a viral capsid sequence. In some embodiments, the nucleic acid comprises a synthetic 5′ UTR in combination with a non-synthetic 3′ UTR.

In some embodiments, the UTR can also include at least one translation enhancer nucleic acid, translation enhancer element, or translational enhancer elements (collectively, “TEE,” which refers to nucleic acid sequences that increase the amount of polypeptide or protein produced from a polynucleotide. As a non-limiting example, the TEE can include those described in US2009/0226470, incorporated herein by reference in its entirety for this purpose, and others known in the art. As a non-limiting example, the TEE can be located between the transcription promoter and the start codon. In some embodiments, the 5′ UTR comprises a TEE. In one aspect, a TEE is a conserved element in a UTR that can promote translational activity of a nucleic acid such as, but not limited to, cap-dependent or cap-independent translation. In one non-limiting example, the TEE comprises the TEE sequence in the 5′-leader of the Gtx homeodomain protein. See, e.g., Chappell et al., PNAS. 2004. 101:9590-9594, incorporated herein by reference in its entirety for this purpose.

### Poly(A) Regions

Aspects of the present disclosure relate to methods of analyzing RNAs containing one or more polyA tails or polyA regions. A “polyA tail” or “polyA region” refers to a region of an RNA that is downstream, e.g., directly downstream (i.e., 3′), from the open reading frame and/or the 3′ UTR that contains multiple, consecutive adenosine monophosphates. In a linear mRNA, this region typically occurs at the 3′ terminal end of the mRNA, and is typically referred to as a “polyA tail.” Because a circular mRNA has no terminal ends, this region is instead referred to as a polyA region in the context of a circular mRNA. In embodiments relating to analysis of circular mRNA, references to a polyA tail are to be understood as referring to a polyA region of the circular mRNA.

A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo, etc.) the poly(A) tail functions to protect mRNA from enzymatic degradation, e.g., in the cytoplasm, and aids in transcription termination, export of the mRNA from the nucleus, and translation.

As used herein, “polyA-tailing efficiency” refers to the amount (e.g., expressed as a percentage) of mRNAs having polyA tail that are produced by an IVT reaction using an input DNA relative to the total number of mRNAs produced in the IVT reaction using the input DNA. The polyA-tailing efficiency of an IVT reaction may vary, for example depending upon the RNA polymerase used, amount or purity of input DNA used, etc. In some embodiments, the polyA-tailing efficiency of an IVT reaction is greater than 85%, 90%, 95%, or 99.9%. Methods of calculating polyA-tailing efficiency are known, for example by determining the amount of polyA tail-containing mRNA relative to total mRNA produced in an IVT reaction by column chromatography (e.g., oligo-dT chromatography).

In some embodiments, at least 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 99.9% of RNAs in an RNA composition produced by a method described herein comprise a polyA tail. In some embodiments, at least 85%, 86%, 87%, 88%, 89%, 90%, 91%, 92%, 93%, 94%, 95%, 96%, 97%, 98%, 99%, or 99.9% of each RNA in an RNA composition produced by a method described herein comprise a polyA tail. The efficiency (e.g., percentage of polyA tail-containing RNAs in an RNA composition may be measured i) after the IVT reaction and before purification, or ii) after the RNA composition has been purified (e.g., by chromatography, such as oligo-dT chromatography).

Unique polyA tail lengths provide certain advantages to the nucleic acids of the present disclosure. Generally, the length of a polyA tail, when present, is greater than 30 nucleotides in length. In another embodiment, the polyA tail is greater than 35 nucleotides in length (e.g., at least or greater than about 15, 20, 25, 30, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, or 3,000 nucleotides).

In some embodiments, the polyA tail is designed relative to the length of the overall nucleic acid or the length of a particular region of the nucleic acid. This design can be based on the length of a coding region, the length of a particular feature or region or based on the length of the ultimate product expressed from the nucleic acids.

In this context, the polyA tail can be 10, 20, 30, 40, 50, 60, 70, 80, 90, or 100% greater in length than the nucleic acid or feature thereof. The polyA tail can also be designed as a fraction of the nucleic acid to which it belongs. In this context, the polyA tail can be 10, 20, 30, 40, 50, 60, 70, 80, or 90% or more of the total length of the construct, a construct region or the total length of the construct minus the polyA tail. Further, engineered binding sites and conjugation of nucleic acids for PolyA-binding protein can enhance expression.

### EXAMPLES

### Example 1: LC/MS Approaches for Intact Mass Spectrometry of mRNA

To assess attributes of our mRNA therapeutics, a liquid chromatography-mass spectrometry (LC/MS)-based multi-attribute method (MAM) was developed. This method provides insight into mRNA homogeneity, cap integrity, and heterogeneity of the poly adenosine (poly-A) tail while circumventing the need for multiple sample preparation steps.

mRNAs were prepared by T7 RNA polymerase in vitro transcription (IVT) using a deoxyribonucleic acid (DNA) template containing, in order, coding sequences for a 5′ untranslated region (UTR), an open reading frame (ORF), a 3′ UTR, and a poly(A) tail. Transcribed mRNA was desalted and separated from IVT reaction components using liquid chromatography. Mass spectrometry analyses of purified mRNAs were performed on an Agilent 6545XT AdvanceBio quadrupole time-of-flight (Q-TOF) mass spectrometer, and data were analyzed using Agilent MassHunter Qualitative Analysis software.

A single mid-length mRNA (1000-2500 nt) contains a large number of potential deprotonation sites. This presents a substantial barrier to mass spectrometry analysis of longer mRNA, as minimal differences exist between the individual charge states in differentially deprotonated forms of the mRNA. This potential lack of resolution, in combination with the general broad distribution of charge states in longer mRNAs, also reduces the signal intensity of any given deprotonation state of the same mRNA. Additionally, heterogeneity exists in the poly(A) tail lengths of a given mRNA species, such that an mRNA having a given 5′ UTR, ORF, and 3′ UTR may vary in length by +/−10 adenosine residues, adding another layer of complexity to the analysis.

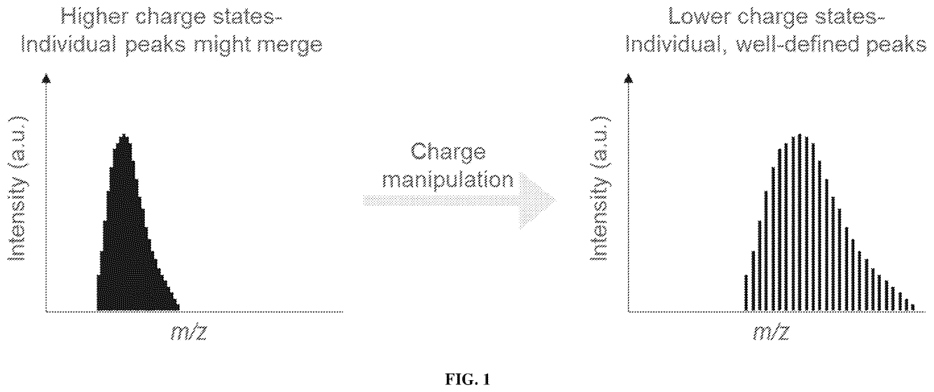

The LC/MS methods provided herein overcome these challenges by using conditions that promote proton-transfer reactions during the ionization process, increasing the probability that a given site is deprotonated, thereby reducing the overall charge state distribution and improving the signal-to-noise ratio of the mRNA analysis (FIG. 1). Reducing the overall charge state distribution in this manner improved the signal-to-noise ratio of mass spectrometry of eluted mRNAs, allowing resolution of distinct ions in mass-to-charge spectra. Using a combination of ammonium bicarbonate/isopropanol in hydrophilic interaction liquid chromatography (HILIC)-based separation, allowed reduction of the overall charge state distribution by about two-fold. The use of ammonium bicarbonate/isopropanol in HILIC separation was particularly more efficient for analysis of mRNAs larger than 1000 nucleotides. After deconvolution, intact mass values of the mRNA formed a Gaussian-like distribution spaced 329 Da apart, which is consistent with the mass of an adenosine residue. Thus, heterogeneity in poly(A) tail lengths of a given mRNA species does not interfere with analysis of mRNA masses. Furthermore, the distribution of poly-A tail lengths observed by analysis of intact mRNAs was compared to the distribution of poly-A tail lengths observed by analysis of RNase H cleavage products containing only the poly-A tail and a portion of the 3′ UTR. The distributions of tail lengths were consistent between analyses of intact mRNAs and analyses of RNase H cleavage products, even though intact mRNAs are at least four times larger than the poly-A tails themselves. The observed consistency between tail length distributions demonstrates that LC/MS methods of analyzing intact mRNAs accurately resolve the masses of mRNAs, including those with different tail poly-A tail lengths.

Next, LC/MS methods were used to characterize 5′ cap integrity of mRNAs. Different levels of collisional activation promoted the formation of multiple distinct cap-related fragments. This group included a fragment consisting of the N7-methylguanosine cap, the triphosphate linkage, and the first two nucleotides of the mRNA. Therefore, mRNAs with different masses due to differences in cap variants can be efficiently differentiated using such LC/MS methods.

### Example 2: Preparation of mRNA for HILIC LC/MS Analysis

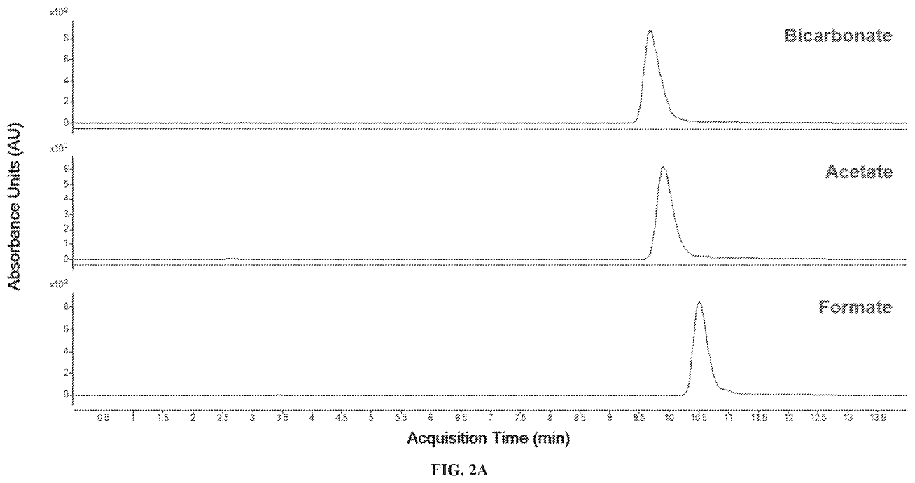

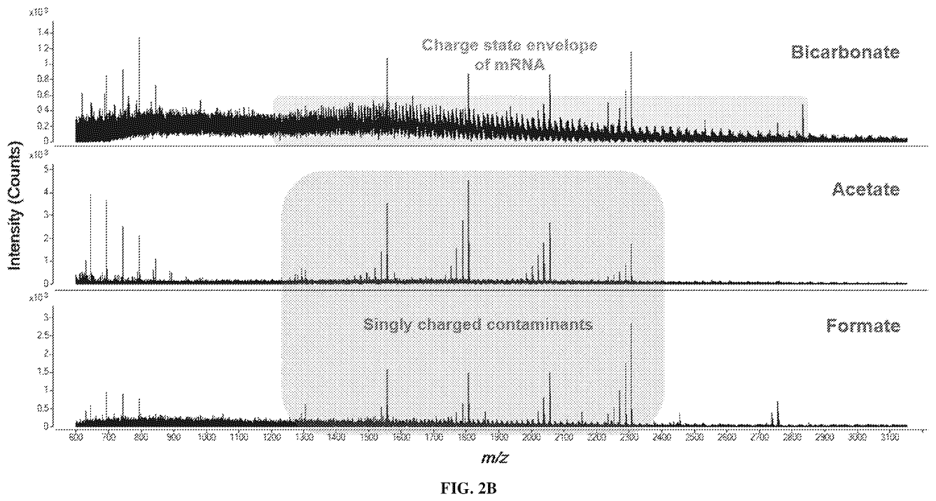

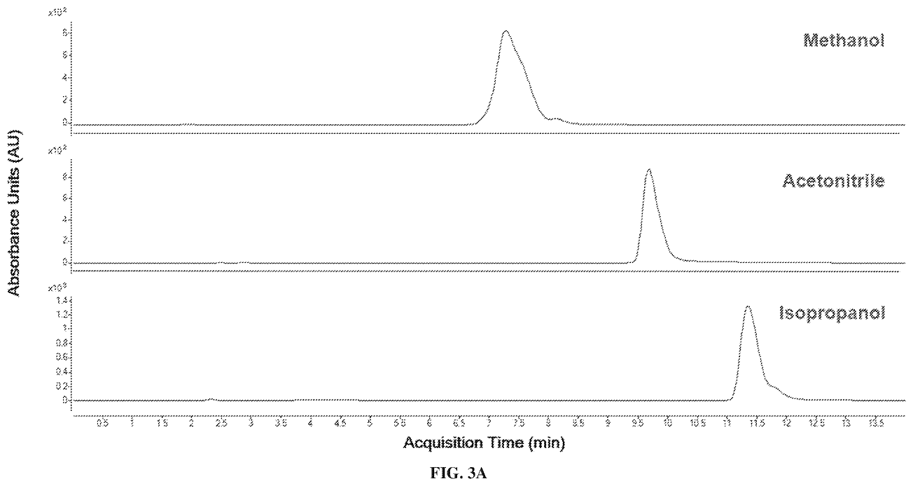

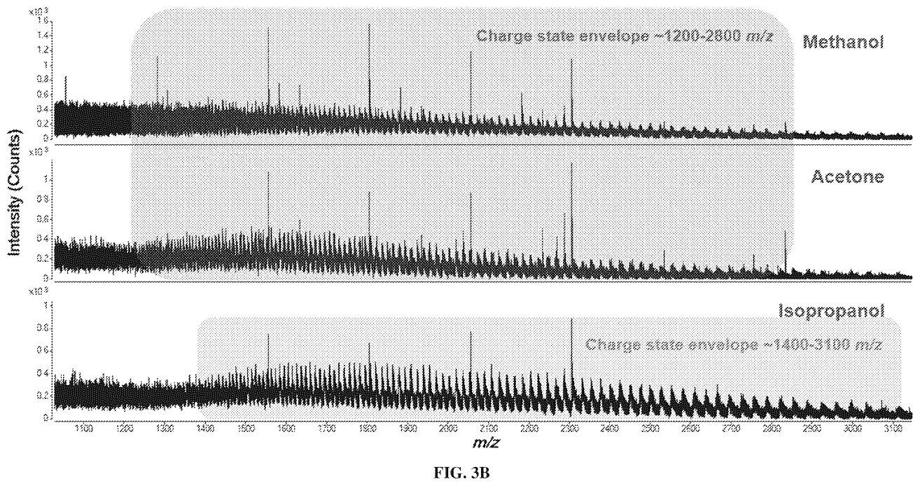

Mobile phases containing a volatile salt, organic solvent, and one or more ion pairing agents were prepared for purification of nucleic acids by hydrophilic interaction chromatography (HILIC)-based methods. Volatile salts tested included ammonium bicarbonate, ammonium acetate, and ammonium formate (FIGS. 2A-2B). Organic solvents tested included methanol, acetonitrile, and isopropanol (FIGS. 3A-3B). Ion pairing agents included octylamine, nonafluoro-tert-butyl alcohol, diethylammonium acetate, dibutylammonium acetate (FIG. 4). The volatile salt and ion pairing agent(s) were dissolved in a solution of the organic solvent in water to prepare a first mobile phase, and in a less concentrated solution of the same organic solvent in water to prepare a second mobile phase. In each mobile phase, a first and second ion pairing agent were combined in a ratio between 1:10 and 10:1, with each ion pairing agent having a final concentration of 0.1 mM-100 mM. A buffer containing the volatile salt was added to the mobile phase to a final volatile salt concentration of 1 mM-100 mM, to promote ionization of the eluted mRNA and reduce the charge state of the mRNA during ionization. Compositions containing mRNA to be analyzed were added to HILIC columns at a temperature between 20° C. and 60° C., and mobile phase was passed through the columns. After elution, purified mRNAs were ionized and analyzed by mass spectrometry (FIGS. 2B, 3B, and 4).

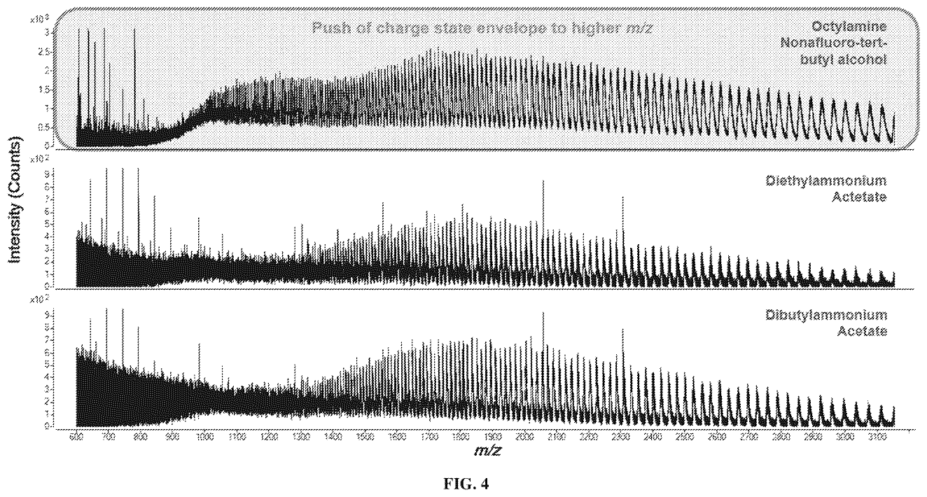

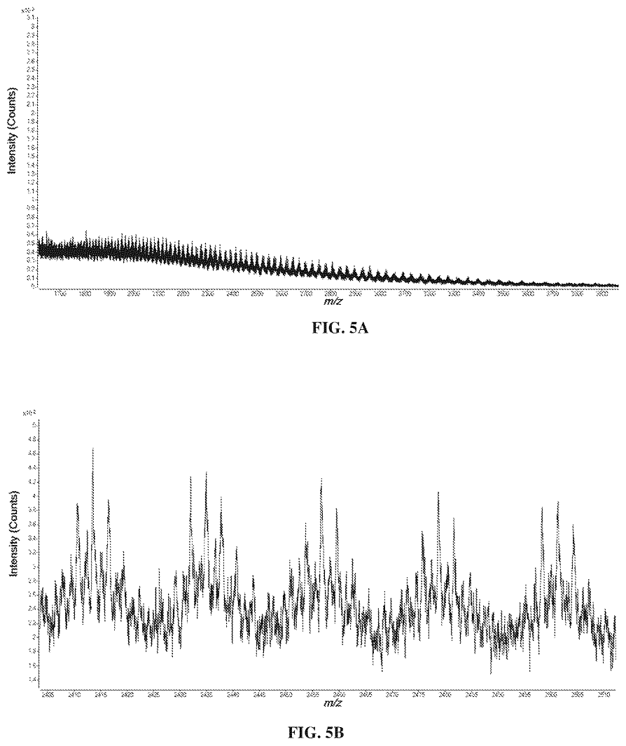

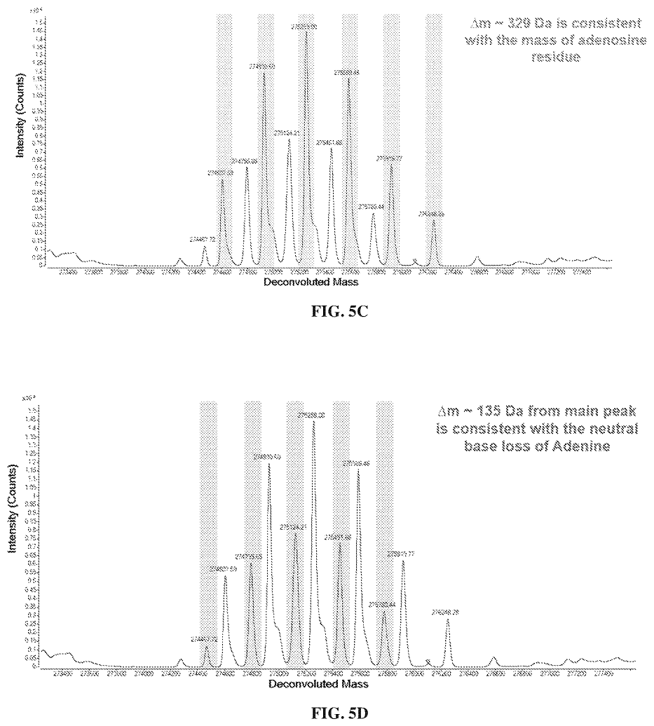

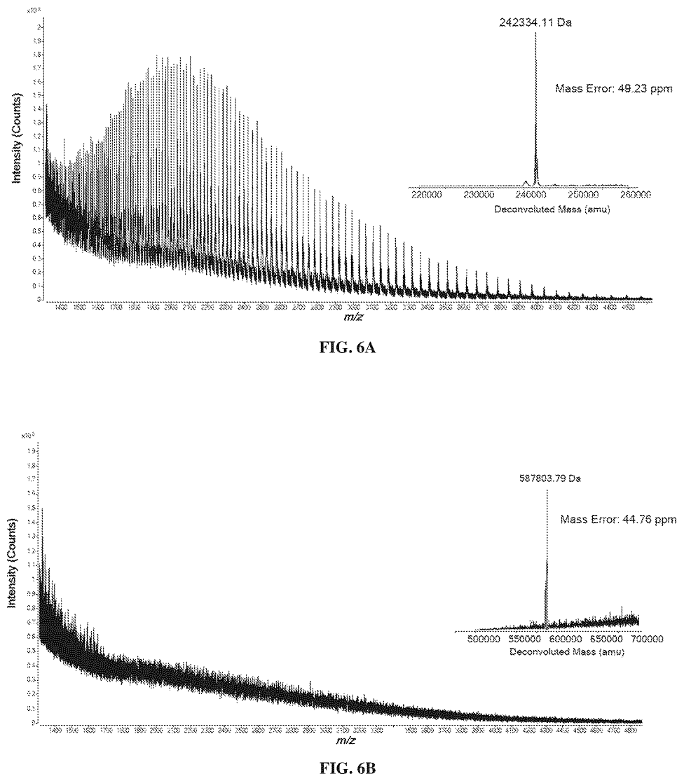

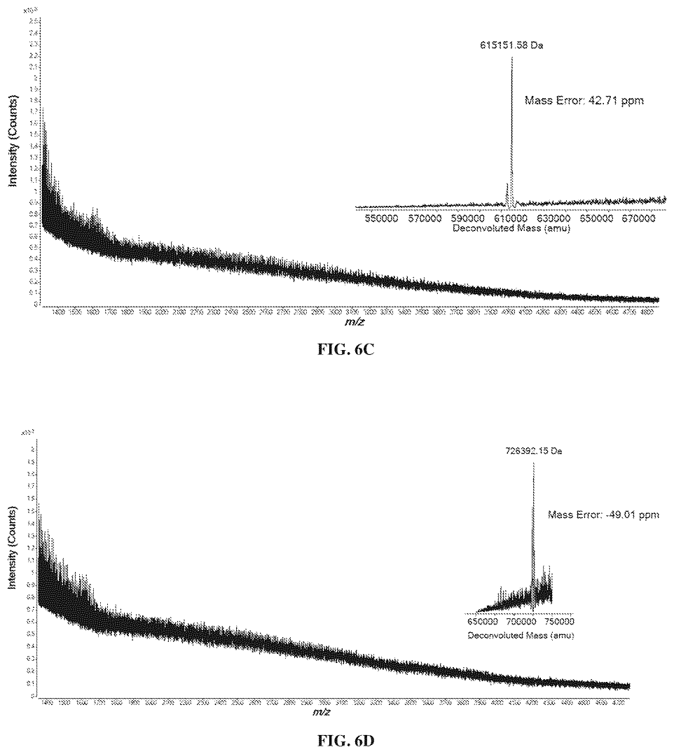

Four mRNAs, each having a different mass, were purified by the HILIC-based method described in the preceding paragraph, using isopropanol as the organic solvent, ammonium bicarbonate (NH₄HCO₃) as the volatile salt, and the combination of octylamine and nonafluoro-tert-butyl alcohol as the ion pairing agents. The first mRNA, having a length of 751 nucleotides, was then analyzed by mass spectrometry. As described in the preceding example, the overall charge state of the first mRNA was reduced, allowing resolution of individual peaks corresponding to distinct ions (FIGS. 5A-5B). Furthermore, deconvolution of spectra generated by analysis of tailed mRNA species produced intact mass values of the mRNAs that formed a Gaussian-like distribution spaced 329 Da apart, consistent with the mass of an adenosine residue (FIGS. 5C-5D). To reduce the heterogeneity in mass resulting from analysis of mRNAs having different poly(A) tail lengths, all four mRNAs were contacted with an RNase H guide that bound to a sequence in the 3′ UTR upstream of the poly(A) tail, and incubated with RNAse H to produce mRNA fragments. Tailless mRNA fragments generated by RNase H cleavage were then ionized and analyzed by mass spectrometry. The four mRNAs had lengths of 751 nucleotides (FIG. 6A), 1822 nucleotides (FIG. 6B), 1894 nucleotides (FIG. 6C), and 2228 nucleotides (FIG. 6D), respectively. The mass of each mRNA was accurately estimated with a margin of error less than 50 parts per million, or 0.005%. These results indicate that HILIC-based methods allow purification and subsequent mass spectrometry analysis of mRNAs that are at least 2000 nucleotides in length. These methods represent a marked improvement over previous methods, which were unable to purify mRNAs longer than 300 nucleotides in length.

### Example 3: Analysis of Circular mRNA by HILIC LC/MS Analysis

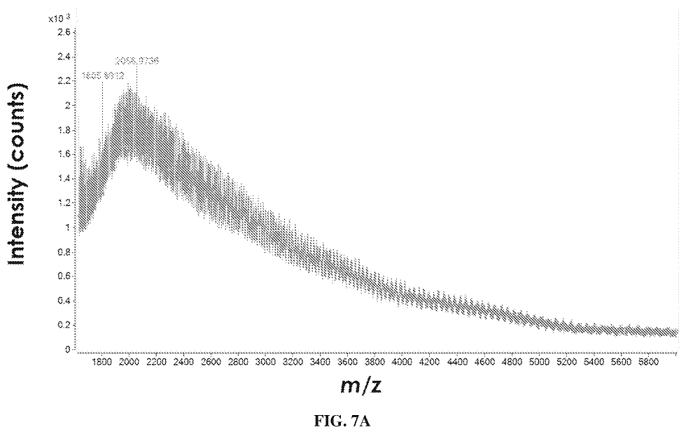

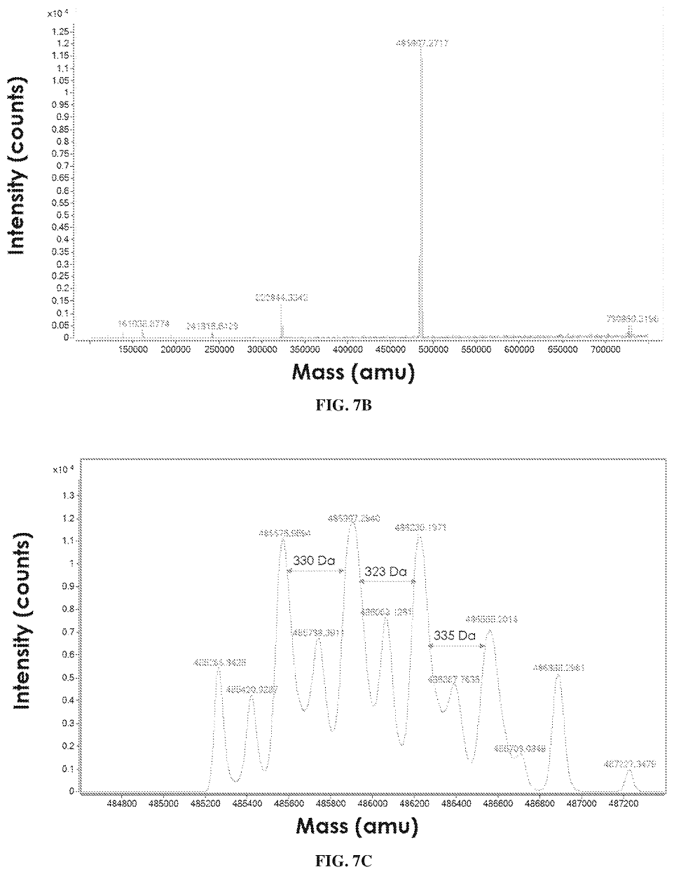

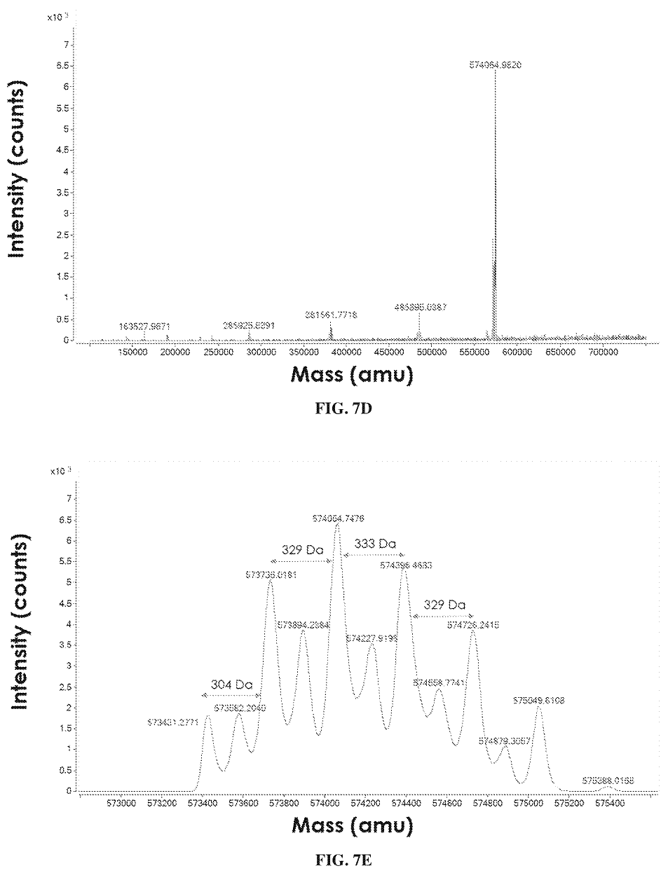

To evaluate the ability of HILIC LC/MS methods to analyze circular mRNAs, two circular mRNAs were prepared, and analyzed by the methods of Example 2, using isopropanol as the organic solvent, ammonium bicarbonate (NH₄HCO₃) as the volatile salt, and the combination of octylamine and nonafluoro-tert-butyl alcohol as the ion pairing agents. As in analysis of linear mRNAs, HILIC LC/MS also produced a mass-to-charge spectrum in which resolution of individual peaks was achieved (FIG. 7A). Deconvolution of spectra for each circular mRNA revealed a set of main peaks corresponding to the theoretical mass of each mRNA (FIGS. 7B, 7D). As with linear mRNAs, each set of peaks followed a Gaussian distribution of individual peaks separated by approximately 329 Da, corresponding to the mass of an adenosine nucleotide, indicating circular mRNAs with slightly different polyA tail lengths.

### EQUIVALENTS AND SCOPE

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in some embodiments, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in some embodiments, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. Each possibility represents a separate embodiment of the present invention.

It should be understood that, unless clearly indicated to the contrary, the disclosure of numerical values and ranges of numerical values in the specification includes both i) the exact value(s) or range specified, and ii) values that are “about” the value(s) or ranges specified (e.g., values or ranges falling within a reasonable range (e.g., about 10% similar)) as would be understood by a person of ordinary skill in the art.

It should also be understood that, unless clearly indicated to the contrary, in any methods disclosed herein that include more than one step or act, the order of the steps or acts of the method is not necessarily limited to the order in which the steps or acts of the method are disclosed.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method of identifying a target mRNA in a mixture, the method comprising:
(i) contacting a stationary phase of a hydrophilic interaction chromatography (HILIC) column with one or more mRNAs;
(ii) detecting a signal corresponding to the retention time of the target mRNA;
(iii) eluting the target mRNA from the HILIC column; and
(iv) determining the mass of the eluted mRNA of (iii) using mass spectrometry.

2. The method of claim 1, further comprising contacting the column with a mobile phase comprising a first solvent solution and a second solvent solution each comprising at least one ion pairing agent, and wherein the first solvent solution further comprises at least about 50% v/v of an organic solvent, such that the target mRNA traverses the column with a retention time that is characteristic of the target mRNA.

3. The method of claim 2, wherein the first solvent solution and second solvent solution each comprise at least two ion pairing agents in a molar ratio of between about 1:10 to about 10:1, optionally wherein the first and/or second solvent solution are in a molar ratio between about 1:4 to about 4:1, about 1:5 to about 5:1, about 1:5 to about 5:1, about 1:3 to about 3:1, about 1:2 to about 2:1, or about 1:1.5 to about 1.5:1, optionally wherein the at least two ion pairing agents in the first and/or second solvent solution are in a 1:1 molar ratio.

4. The method of claim 2 or claim 3, wherein the at least one ion pairing agent in the first and/or second solvent solution is selected from the group consisting of a trietheylammonium salt, tributylammonium salt, hexylammonium salt, dibutylammonium salt, tetrapropylammonium salt, dodecyltrimethylammonium salt, tetra(decyl)ammonium salt, dihexylammonium salt, dipropylammonium salt, myristyltrimethylammonium salt, tetraethylammonium salt, tetraheptylammonium salt, tetrahexylammonium salt, tetrakis(decyl)ammonium salt, tetramethylammonium salt, tetraoctylammonium salt, and tetrapentylammonium salt, optionally wherein the triethylammonium salt is triethylammonium acetate, the tributylammonium salt is tetrabutylammonium phosphate or tetrabutylammonium chloride, the hexylammonium salt is hexylammonium acetate, the dibutylammonium salt is dibutylammonium acetate, the tetrapropylammonium salt is dodecyltrimethylammonium chloride, the tetra(decyl)ammonium salt is tetra(decyl)ammonium bromide, the dihexylammonium salt is dihexylammonium acetate, the dipropylammonium salt is dipropylammonium acetate, the myristyltrimethylammonium salt is myristyltrimethylammonium bromide, the tetraethylammonium salt is tetraethylammonium bromide, the etraheptylammonium salt is tetraheptylammonium bromide, the tetrahexylammonium salt is tetrahexylammonium bromide, the tetrakis(decyl)ammonium salt is tetrakis(decyl)ammonium bromide, the tetramethylammonium salt is tetramethylammonium bromide, the tetraoctylammonium salt is tetraoctylammonium bromide, and/or the tetrapentylammonium salt is tetrapentylammonium bromide.

5. The method of any one of claims 2-4, wherein the first solvent solution and the second solvent solution each comprise at least two ion pairing agents, wherein the at least two ion pairing agents are (i) octylamine and nonafluoro-tert-butyl alcohol; (ii) octylamine and diethylammonium acetate; (iii) octylamine and dibutylammonium acetate; or (iv) diethylammonium acetate and imidazole.

6. The method of any one of claims 2-5, wherein the concentration of each of the at least one ion pairing agents in the first solvent solution and/or the second solvent solution ranges from about 10 mM-20 M, 20 mM-15 M, 30 mM-12 M, 40 mM-10 M, 50 mM-8 M, 75 mM-5 M, 100 mM-2.5 M, 125 mM-2 M, 150 mM-1.5 M, 175 mM-1 M, or 200 mM-500 mM,
optionally wherein the concentration of each of the at least one ion pairing agents in the first solvent solution and/or the second solvent solution ranges from about 10 mM-1M, 40 mM-300 mM, 50 mM-500 mM, 75 mM-400 mM, 100 mM-300 mM, 200-300 mM, 200-250 mM, or 250-300 mM.

7. The method of any one of claims 2-6, wherein the first solvent solution comprises about 50% to about 95%, about 55% to about 90%, about 60% to about 85%, about 65% to about 80%, or about 70% v/v to about 75% v/v of the organic solvent,
optionally wherein the first solvent solution comprises about 50%, about 60%, about 70%, about 80%, or about 90% v/v of the organic solvent.

8. The method of any one of claims 2-7, wherein the organic solvent in the first solvent solution is selected from the group consisting of polar aprotic solvents, C₁₋₄ alkanols, C₁₋₆ alkanediols, and C₂₋₄ alkanoic acids.

9. The method of any one of claims 2-8, wherein the organic solvent in the first solvent solution is selected from the group consisting of acetonitrile, methanol, ethanol, isopropanol, acetone, propanol, tetrahydrofuran, dimethyl sulfoxide, dimethylformamide, and hexylene glycol.

10. The method of any one of claims 2-9, wherein the pH of the first solvent solution and/or the second solvent solution is between about pH 6.5 and pH 9.0.

11. The method of any one of claims 2-10, wherein the volume percentage of the first solvent solution and volume percentage of the second solvent solution in the mobile phase are each between 0% and 100%.

12. The method of any one of claims 2-11, wherein the ratio of the first solvent solution to the second solvent solution is held constant during elution of the mRNA.

13. The method of any one of claims 2-12, wherein the ratio of the first solvent solution to the second solvent solution is increased or decreased during elution of the mRNA.

14. The method of any one of claims 2-13, wherein the concentration of each ion pairing agent in the mobile phase is held constant during elution of the mRNA.

15. The method of any one of claims 2-14, wherein the concentration of one or more ion pairing agents in the mobile phase is increased or decreased during elution of the mRNA.

16. The method of any one of claims 2-15, wherein the eluting is gradient or isocratic with respect to the concentration of the organic solvent.

17. The method of any one of claims 1-16, wherein each of the first and second solvent solutions comprises one or more volatile salts.

18. The method of claim 17, wherein the at least one volatile salt in the first and/or second solvent solution is selected from the group consisting of formic acid, acetic acid, trifluoroacetic acid, ammonium formate, ammonium acetate, ammonium hydroxide, triethylamine acetate, triethylamine formate, diethylamine acetate, diethylamine formate, piperidine acetate, piperidine formate, ammonium bicarbonate, borate, hydride, 4-methylmorpholine, 1-methylpiperidine, pyrrolidine acetate, and pyrrolidine formate.

19. The method of claim 17 or 18, wherein the concentration of each of the at least one volatile salts in the first solvent solution and/or the second solvent solution ranges from about 10 mM-20 M, 20 mM-15 M, 30 mM-12 M, 40 mM-10 M, 50 mM-8 M, 75 mM-5 M, 100 mM-2.5 M, 125 mM-2 M, 150 mM-1.5 M, 175 mM-1 M, or 200 mM-500 mM,
optionally wherein the concentration of each of the at least one volatile salts in the first solvent solution and/or the second solvent solution ranges from about 10 mM-1M, 40 mM-300 mM, 50 mM-500 mM, 75 mM-400 mM, 100 mM-300 mM, 200-300 mM, 200-250 mM, or 250-300 mM.

20. The method of any one of claims 1-19, wherein the column is an analytical column, or a preparative column.

21. The method of any one of claims 1-20, wherein the stationary phase comprises particles.

22. The method of claim 21, wherein the particles have a diameter of about 2 μm-about 10 μm, about 2 μm-about 6 μm, or about 4 μm.

23. The method of claim 21 or 22, wherein the particles are porous resin particles, optionally wherein the particles comprise pores having a diameter of about 500 Å to about 5000 Å, about 800 Å to about 3000 Å, or about 1000 Å to about 2000 Å.

24. The method of any one of claims 1-23, wherein the stationary phase is hydrophilic or comprises hydrophilic functional groups.

25. The method of any one of claims 1-24, wherein the column has a temperature from about 20° C. to about 60° C.

26. The method of any one of claims 1-25, wherein the method has a run time of between about 10 minutes and about 30 minutes.

27. The method of any one of claims 1-26, wherein the target mRNA is present in a composition added to the column in an amount ranging from about 0.05 mg/mL to about 1 mg/mL, optionally wherein the amount is 0.1 mg/mL.

28. The method of any one of claims 1-27, wherein determining the mass of the eluted mRNA using mass spectrometry comprises using MALDI and/or ESI to ionize the mRNA, followed by TOF mass spectrometry to analyze the ionized mRNA.

29. The method of any one of claims 1-28, wherein the target mRNA is single-stranded.

30. The method of any one of claims 1-29, wherein the target mRNA comprises:
(i) 5′ and 3′ untranslated regions (UTRs);
(ii) a 5′ cap, optionally wherein the 5′ cap is a 7-methylguanosine cap or a 7-methylguanosine group analog; and
(iii) a 3′ polyadenosine (poly(A)) tail.

31. The method of claim 30, wherein the target mRNA is a linear mRNA.

32. The method of any one of claims 1-29, wherein the target mRNA is a circular mRNA.

33. The method of claim 32, wherein the circular mRNA comprises an internal ribosome entry site (IRES).

34. The method of claim 32 or 33, wherein the circular mRNA comprises in 5′ to 3′ order, a 5′ untranslated region (UTR), an IRES, an open reading frame encoding a protein, and a 3′ untranslated region.

35. The method of claim 34, wherein the circular RNA further comprises a poly(A) region.

36. The method of claim 35, wherein the poly(A) region is between the 5′ UTR and the IRES.

37. The method of claim 35 or 36, wherein the poly(A) region is between the open reading frame and the 3′ UTR.

38. The method of any one of claims 1-37, wherein the mRNA is an in vitro transcribed (IVT) mRNA.

39. The method of any one of claims 1-38, wherein the mRNA encodes a vaccine antigen or therapeutic polypeptide.

40. The method of any one of claims 1-39, wherein the target mRNA has a length between 300-500 nucleotides, 500-1000 nucleotides, 1000-1500 nucleotides, 1500-2000 nucleotides, 2000-2500 nucleotides, 2500-3000 nucleotides, 3000-3500 nucleotides, 3500-4000 nucleotides, 4000-4500 nucleotides, or 4500-5000 nucleotides.
